(19) United States
(12) Patent Application Publication
(10) Pub. No.: US 2018/0200324 A1
(43) Pub. Date: Jul. 19, 2018

(54) ARTIFICIAL PEPTIDES AND USE THEREOF FOR GLYCOGEN STORAGE DISORDERS

(71) Applicant: HADASIT MEDICAL RESEARCH SERVICES AND DEVELOPMENT LTD., Jerusalem (IL)

(72) Inventors: Or KAKHLON, Jerusalem (IL); Amit MICHAELI, Jerusalem (IL)

(73) Assignee: HADASIT MEDICAL RESEARCH SERVICES AND DEVELOPMENT LTD., Jerusalem (IL)

(21) Appl. No.: 15/743,056

(22) PCT Filed: Jul. 21, 2016
(86) PCT No.: PCT/IL2016/050800

§ 371 Date: Jan. 9, 2018

Related U.S. Application Data
(60) Provisional application No. 62/195,833, filed on Jul. 23, 2015.

(51) Int. Cl. A61K 38/07; C07K 5/113; C07K 5/103; A61P 3/08

(52) U.S. Cl. CPC A61K 38/07; A61P 3/08; C07K 5/101; C07K 5/1021

(57) ABSTRACT

The present invention discloses a peptide capable of stabilizing mutation-induced GBE1 protein destabilization, conjugates comprising same and uses thereof for the treatment of diseases and disorders associate with glycogen storage.

### FIELD OF THE INVENTION

The present invention relates to artificial peptides, preparation and uses thereof for treatment of glycogen storage disorders.

### BACKGROUND

Glycogen is a compact polymer of alpha-1,4-linked glucose units regularly branched with alpha-1,6-glucosidic bonds, serving as the main carbohydrate store and energy reserve across many phyla.

In eukaryotes, glycogenin initiates synthesis of the linear glucan chain which is elongated by glycogen synthase (GYS), functioning in concert with glycogen branching enzyme (GBE) to introduce side chains.

Mutations in the human GBE1 (hGBE1) gene (chromosome 3p12.3) cause the autosomal recessive glycogen storage disorder type IV (GSDIV), which is characterized by the deposition of an amylopectin-like polysaccharide that has fewer branch points, longer outer chains and poorer solubility than normal glycogen. GSDIV is an extremely heterogeneous disorder with variable onset age and clinical severity, including a late-onset allele variant—adult polyglucosan body disease (APBD)—a neurological disorder affecting mainly the Ashkenazi Jewish population.

US 2016/0030375 and US 20140/288175 disclose methods for treating glycogen storage disease, primarily GSD II, by using a composition that includes ketogenic odd carbon fatty acids.

US 2015/0273016 discloses gene therapy for glycogen storage diseases, including, GSDIV by delivering a nucleic acid encoding a transcription factor EB (TFEB) gene into a subject in need thereof.

US 2011/0306663 discloses a method of treating adult polyglucosan body disorder (APBD) by using triheptanoin (C7TG), optionally, mixed in with one or more food products for oral consumption.

There is an unmet need for improved treatments of disorders associated with glycogen storage, including, GSDIV and APBD.

### SUMMARY

The present invention discloses a peptide capable of stabilizing mutation-induced GBE1 protein destabilization, conjugates comprising same and uses thereof for the treatment of diseases and disorders associate with glycogen storage. It has been shown in the current disclosure and published by the inventors and their co-workers (Froese et al., Hum. Mol. Genet., 24(20): 5667-5676, 2015; first published on line on Jul. 21, 2015) for the first time, that GBE1 mutation can result in protein destabilization, lending support to the emerging concept, among many metabolic enzymes, that mutation-induced protein destabilization could play a causative role in disease pathogenesis. Thus, the present invention is based in part on the unexpected finding that the p.Y329S of hGBE1 mutation, which is commonly associated with APBD, results in protein destabilization. Based on these findings, peptides were designed in silico and their ability to rescue hGBE1 from the p.Y329S-associated protein destabilization was examined. Surprisingly, it was found that use of a small peptide as chaperone, such as, the LTKE peptide in APBD, can stabilize GBE1 mutant and rescue GBE1 mutant activity to 10-15% of wild-type.

Without being bound by any theory or mechanism, it is proposed that the LTKE peptide binds to mutant GBE1 possibly in a co-translational manner, akin to the binding of cellular chaperones to nascent polypeptide chains during protein synthesis, thereby allowing peptide access to the mutation induced cavity as the protein is being folded in the cell. In some metabolic disorders (e.g. lysosomal storage diseases), a 10-15% recovery of mutant enzyme activity was sufficient to ameliorate disease phenotypes.

Some of the advantages of using small peptides for therapy include, but are not limited to, low toxicity, low production costs and the possibility of incorporation into gene therapy, which is particularly useful in chronic conditions, such as, APBD.

In some embodiments, there is provided an artificial peptide comprising amino acid sequence Leu-Thr-Lys-Glu (SEQ ID NO:1).

In some embodiments, the artificial peptide is consisting of the amino acid sequence set forth in SEQ ID NO: 1.

In some embodiments, there is provided a conjugate comprising the artificial peptide disclosed herein and a moiety linked thereto, optionally via a spacer, wherein the moiety is selected from the group consisting of a fluorescent probe, a photosensitizer, a chelating agent and a therapeutic agent. Each possibility represents a separate embodiment of the present invention.

In some embodiments, the spacer is selected from the group consisting of a natural or non-natural amino acid, a short peptide having no more than 8 amino acids and a C1-C25 alkyl. Each possibility represents a separate embodiment of the present invention.

In some embodiments, said moiety is a fluorescent probe.

In some embodiments, said fluorescent probe is selected from the group consisting of BPheide taurine amide (BTA), fluorenyl isothiocyanate (FITC), dansyl, rhodamine, eosin and erythrosine. Each possibility represents a separate embodiment of the present invention.

In some embodiments, the peptide within the conjugate is consisting of the amino acid sequence set forth in SEQ ID NO:1.

In some embodiments, there is provided a pharmaceutical composition comprising the artificial peptide disclosed herein and a pharmaceutically acceptable carrier.

In some embodiments, there is provided a pharmaceutical composition comprising the conjugate disclosed herein.

In some embodiments, there is provided a use of a pharmaceutical composition comprising an artificial peptide comprising the amino acid sequence set forth in SEQ ID NO: 1 for the treatment of a disease or disorder associated with glycogen storage. Each possibility represents a separate embodiment of the present invention.

In some embodiments, the disease or disorder is glycogen storage disorder type IV (GSDIV) or late-onset adult polyglucosan body disease (APBD). Each possibility represents a separate embodiment of the present invention.

In some embodiments, the disease or disorder is APBD.

In some embodiments, there is provided use of a pharmaceutical composition comprising a conjugate comprising an artificial peptide comprising the amino acid sequence set forth in SEQ ID NO: 1 and a moiety linked thereto, optionally via a spacer, wherein the moiety is selected from the group consisting of a fluorescent probe, a photosensitizer, a chelating agent and a therapeutic agent. Each possibility represents a separate embodiment of the present invention.

In some embodiments, there is provided a method of treating disease or disorder associated with glycogen storage in a subject in need thereof, the method comprising administering to said subject a pharmaceutical composition comprising an artificial peptide comprising the amino acid sequence set forth in SEQ ID NO: 1.

In some embodiments, there is provided a method of treating disease or disorder associated with glycogen storage in a subject in need thereof, the method comprising administering to said subject a pharmaceutical composition comprising a conjugate comprising an artificial peptide comprising the amino acid sequence set forth in SEQ ID NO: 1 and a moiety linked thereto, optionally via a spacer, wherein the moiety is selected from the group consisting of a fluorescent probe, a photosensitizer, a chelating agent and a therapeutic agent. Each possibility represents a separate embodiment of the present invention.

In some embodiments, the subject is human.

In some embodiments, treating comprising any one or more of preventing the onset of said disease or disorder, preventing or reducing the progression of said disease or disorder and reducing the pathology and/or symptoms associated with said disease or disorder. Each possibility represents a separate embodiment of the present invention.

In addition to the exemplary aspects and embodiments described above, further aspects and embodiments will become apparent by reference to the figures and by study of the following detailed descriptions.

### BRIEF DESCRIPTION OF THE DRAWINGS

FIG. 1A shows the crystal structure of hGBE1.

FIG. 1B shows the crystal structure of hGBE1 from a different angle.

FIG. 1C shows a structural overlay of hGBE1 with reported branching enzyme structures from O. sativa SBE1.

FIG. 1D shows domains comparison of hGBE1, O. sativa SBE1 and M. tuberbulosis GBE.

FIG. 2A shows the chemical structures of acarbose (ACR) and (Glc₇).

FIG. 2B shows surface representation of hGBE1 indicating the bound oligosaccharides.

FIG. 2C shows ACR binding cleft at the interface of the helical segment, CBM48 and catalytic domain. Shown in sticks are ACR and its contact protein residues. Inset, 2Fo-Fc electron density for the modelled ACR.

FIG. 2D shows sequence alignment of the ACR binding residues of hGBE1 (underlined) in human DNA (SEQ ID NOS: 8 and 14) and DNA from various species (SEQ ID NOS: 9-13 and 15-19).

FIG. 2E shows surface representation of the hGBE1-Glc₇ complex to model the two GBE reaction steps. Left panel is overlaid with a decasaccharide ligand and TIM barrel loops from the B. amyloliquefaciens and B. licheniformis chimeric amylase structure (PDB code 1e3z) to highlight the broader active site cleft in hGBE1 due to the absence of these amylase loops. Right panel is overlaid with maltotriose from pig pancreatic α-amylase (PDB code lua3), as well as the β4-α4 loop from O. sativa SBE1 and M. tuberculosis GBE structures, which is disordered in hGBE1.

FIG. 2F shows close-up view of the hGBE1 active site barrel that harbors the conserved residues (sticks) of the “−1” subsite.

FIG. 3A shows mapping of disease-associated missense mutation sites on the hGBE1 structure underlines their prevalence in the central catalytic core. Inset, view of the hGBE1 sites showing four missense mutation sites which could be involved in binding a glucan chain, indicated by an overlaid decasacharide ligand from the 1e3z structure.

FIG. 3B shows structural environment of representative mutation sites compared to wild type.

FIG. 3C shows structural environment of representative mutation sites compared to wild type.

FIG. 3D shows structural environment of representative mutation sites compared to wild type.

FIG. 3E shows structural environment of representative mutation sites compared to wild type.

FIG. 4A shows conserved domain in hGBE1 from human DNA (SEQ ID NO: 20) and DNA of various species (SEQ ID NOS: 21-29), indicating that Tyr329 is highly conserved across various GBE orthologs.

FIG. 4B is an SDS-PAGE of affinity purified hGBE1 WT and p.Y329S, exhibiting much reduced level of soluble mutant protein.

FIG. 4C is structural analysis of Tyr329 and its neighbourhood revealing a number of hydrophobic interactions which are removed by its substitution with serine.

FIG. 4D shows that Tyr329 (left panel) is accessible to the protein exterior, and its mutation to Ser329 (right panel) creates a cavity (circled).

FIG. 5A shows root mean squared deviations (RMSD) from the backbone as a representation of structural stability in silico.

FIG. 5B shows the molecular mechanics force field calculated binding free energy contributions of individual amino acids in the tetra-peptide LTKE, indicating that the Leu N-terminus contributes more than half of total binding free energy.

FIG. 5C is a homology model of hGBE1-Y329S in complex with the LTKE peptide at the Ser329ₘᵤₜₐₙₜ cavity.

FIG. 5D is a close-up view of the LTKE peptide, where the side-chain of the N-terminal leucine (Leuᵢ) residue fills the cavity.

FIG. 5E is view of the predicted hydrogen bonds (in dotted lines) within the LTKE-bound hGBE1-Y329S model.

FIG. 6A shows intracellular peptide uptake, determined by flow cytometry, of FITC-labeled LTKE peptides in PBMCs isolated from APBD patients incubated at 37° C. (filled squares) or 4° C. (empty squares).

FIG. 6B is an SDS-PAGE and immunoblotting with anti-GBE1 and anti-α-tubulin (loading control) antibodies of isolated PBMCs from an APBD patient (Y329S), or a control subject (WT), incubated overnight with or without the peptides indicated (20 μM).

FIG. 6C shows GBE activity in isolated PBMCs from healthy subjects (health control; x) or PBMCs from an APBD patient (i.e. having the Y329S mutation), untreated (patient; diamond) or treated with LTKE (SEQ ID NO: 1; squares) or EKTL (SEQ ID NO: 2; triangle).

FIG. 6D shows standard curve showing displacement of solid phase FITC by soluble LTKE-FITC.

FIG. 6E shows FITC-labelled peptide competition experiment.

FIG. 7 shows constructs of hGBE1 attempted for recombinant expression, where constructs marked black gave milligram quantities of soluble protein when expressed in liter scale.

FIG. 8A shows binding mode of maltoheptaose in the hGBE1-Glc7 structure with the orientation of acarbose shown as an overlay from the hGBE1-ACR structure.

FIG. 8B shows comparison of oligosaccharide binding mode of CBM48 modules from the O. sativa SBE1 structure complexed with maltopentaose (PDB 3vu2).

FIG. 8C shows comparison of oligosaccharide binding mode of CBM48 modules from the O. sativa SBE1 structure complexed with acarbose.

FIG. 8D shows A. Niger GH15 glucoamylase structure complexed with cyclodextrin.

FIG. 8E shows the three CBM48 modules superimposed.

FIG. 9A shows structural superposition of human pancreatic α-amylase bound with an acarbose-derived hexasaccharide (PDB 1xh0, purple), a chimeric α-amylase complex from B. amyloliquefaciens and B. licheniformis bound with a decasaccharide (1e3z), B. stearothermophilus TRS40 neopullulanase bound with maltotetraose (1j0j), P. haloplanctis α-amylase bound with a heptasaccharide (1g94), and pig pancreatic α-amylase bound with maltotriose (lua3).

FIG. 9B shows structural superposition of hGBE1-apo (4bzy, black) overlaid with 1e3z and lua3 structures.

FIG. 10A is alignment of sequences constituting the four conserved motifs among the GH13 family of enzymes from human (SEQ ID NOS: 30, 36, 42 and 48), O. sativa (RiceBE; SEQ ID NOS: 31, 37, 43 and 49), M. tuberculosis (Mtu GBE; SEQ ID NOS: 32, 38, 44, 50) and E. coli (SEQ ID NOS: 33, 39, 45 and 51), human pancreas α-amylase (1cpu; SEQ ID NOS: 34, 40, 46 and 52) and the chimeric α-amylase complex from B. amyloliquefaciens and B. licheniformis (1e3z; SEQ ID NOS: 35, 41, 47 and 53), highlighting the strictly conserved seven amino acids that form the “−1” subsite.

FIG. 10B is sequence alignment of a ˜30 amino acid stretch that is conserved among branching enzyme orthologues (SEQ ID NOS: 54-57), but not among amylases within the GH13 family (SEQ ID NOS: 58 and 59).

FIG. 11 presents the two-step catalytic mechanism proposed for the hGBE1 branching reaction, sugar subsites are indicated by arcs, nucleophilic attacks by grey arrows, and hydrogen bonds by dashed lines.

FIG. 12 shows amino acid conservation of GBE1 missense mutation sites, identical amino acids, and conserved in human DNA (SEQ ID NO: 60) and DNA of various species (SEQ ID NOS: 61-68).

FIG. 13 shows control peptides binding conditions.

### DETAILED DESCRIPTION

The present invention discloses an artificial peptide, produced based on calculations in silico, capable of stabilizing mutation-induced GBE1 protein destabilization, conjugates comprising same and uses thereof for the treatment of diseases and disorders associate with glycogen storage.

Glycogen branching enzyme (GBE; also known as 1,4-glucan:1,4-glucan 6-glucanotransferase) transfers alpha-1,4-linked glucose units from the outer ‘non-reducing’ end of a growing glycogen chain into an alpha-1,6 position of the same or neighbouring chain, thereby creating glycogen branches. GYS and GBE define the globular and branched structure of glycogen which increases its solubility by creating a hydrophilic surface and regulates its synthesis by increasing the number of reactive termini for GYS-mediated chain elongation.

Glycogen branching enzyme 1 (GBE1) plays an essential role in glycogen biosynthesis by generating α-1,6-glucosidic branches from α-1,4-linked glucose chains, to increase solubility of the glycogen polymer. Mutations in the GBE1 gene lead to the heterogeneous early-onset glycogen storage disorder type IV (GSDIV) or the late-onset adult polyglucosan body disease (APBD).

GBE is classified as a carbohydrate-active enzyme (http://www.cazy.org), and catalyzes two reactions presumably within a single active site. In the first reaction (amylase-type hydrolysis), GBE cleaves, every 8-14 glucose residues of a glucan chain, an α-1,4-linked segment of >6 glucose units from the non-reducing end. In the second reaction (transglucosylation), it transfers the cleaved oligosaccharide (donor′), via an α-1,6-glucosidic linkage, to the C6 hydroxyl group of a glucose unit (acceptor′) within the same chain (intra-) or onto a different neighboring chain (inter-). The mechanistic determinants of the branching reaction, e.g. length of donor chain, length of transferred chain, distance between two branch points, relative occurrence of intra- vs inter-chain transfer, variation among organisms, remain poorly understood.

Almost all sequence-annotated branching enzymes, including those from diverse organisms, belong to the GH13 family of glycosyl hydrolases (also known as the α-amylase family)(5), and fall either into subfamily 8 (eukaryotic GBEs) or subfamily 9 (prokaryotic GBEs) (15). The GH13 family is the largest glysoyl hydrolase family, comprised of amylolytic enzymes (e.g. amylase, pullulanase, cyclo-maltodextrinase, cyclodextrin glycosyltransferase) that carry out a broad range of reactions on α-glycosidic bonds, including hydrolysis, transglycosylation, cyclization and coupling. These enzymes share a (β/α)8 barrel domain with an absolutely conserved catalytic triad (Asp-Glu-Asp) at the C-terminal face of the barrel. In several GH13 enzymes this constellation of three acidic residues functions as the nucleophile (Asp357, hGBE1 numbering hereinafter), proton donor (Glu412), and transition state stabilizer (Asp481) in the active site. To date, crystal structures available from GH13-type GBEs from plant and bacteria have revealed an overall conserved architecture, however, no mammalian enzyme has yet been crystallized. In this study, we determined the crystal structure of hGBE1 in complex with oligosaccharides, investigated the structural and molecular bases of disease-linked missense mutations, and provided proof-of-principle rescue of mutant hGBE1 activity by rational peptide design.

Inherited mutations in the human GBE1 (hGBE1) gene (chromosome 3p12.3) cause the autosomal recessive glycogen storage disorder type IV (GSDIV). GSDIV constitutes about 3% of all GSD cases, and is characterized by the deposition of an amylopectin-like polysaccharide that has fewer branch points, longer outer chains and poorer solubility than normal glycogen. This malconstructed glycogen (termed polyglucosan), presumably the result of GYS activity outpacing that of mutant GBE, accumulates in most organs including liver, muscle, heart, and the central and peripheral nervous systems, leading to tissue and organ damage, and cell death. GSDIV is an extremely heterogeneous disorder with variable onset age and clinical severity, including: a classical hepatic form in neonates and children that progresses to cirrhosis (Andersen disease), a neuromuscular form classified into four subtypes (perinatal, congenital, juvenile, adult-onset), as well as a late-onset allele variant—adult polyglucosan body disease (APBD).

Crystallization of human GBE1 in the apo form, and in complex with a tetra- or hepta-saccharide, as disclosed herein, revealed a conserved amylase core that houses the active center for the branching reaction, and harbors almost all GSDIV and APBD mutations. A non-catalytic binding cleft, proximal to the site of the common APBD mutation p.Y329S, was found to bind the tetra- and hepta-saccharides, and may represent a higher-affinity site employed to anchor the complex glycogen substrate for the branching reaction. Expression of recombinant GBE1-p.Y329S resulted in drastically-reduced protein yield and solubility compared to wild-type, suggesting this disease allele causes protein misfolding and may be amenable to small molecule stabilization. Thus, a structural model of GBE1-p.Y329S was generated and peptides which can stabilize the mutation were designed in silico.

In some embodiments, there is provided an artificial peptide comprising an amino acid sequence selected from the group of LTKE (SEQ ID NO:1); EKEPFEMFM (SEQ ID NO: 3); SSKI (SEQ ID NO: 4) and MKWE (SEQ ID NO: 5); KSLRKW (SEQ ID NO: 6); and SDHRKMYEGR (SEQ ID NO: 7). Each possibility represents a separate embodiment of the present invention.

The term “amino acid” as used herein refers to an organic compound comprising both amine and carboxylic acid functional groups, which may be either a natural or non-natural amino acid.

The term “peptide” as used herein refers to a polymer of amino acid residues. This term may apply to amino acid polymers in which one or more amino acid residue is an artificial chemical analogue of a corresponding naturally occurring amino acid, as well as to naturally occurring amino acid polymers.

The artificial peptide disclosed herein can be optionally modified and/or flanked with additional amino acid residues so long as the peptide retains its stabilizing activity. The particular amino acid sequence(s) flanking the peptide are not limited and may be composed of any kind of amino acids, so long as it does not impair the stabilizing activity of the original peptide.

In general, the modification of one, two, or more amino acids in a protein or a peptide will not influence the function of the protein, and in some cases will even enhance the desired function of the original protein. In fact, modified peptides (i.e., peptides composed of an amino acid sequence in which one, two or several amino acid residues have been modified (i.e., carboxymethylated, biotinylated, substituted, added, deleted or inserted) as compared to an original reference sequence) have been known to retain the biological activity of the original peptide. Thus, in one embodiment, the peptides of the present invention may have both stabilizing activity and an amino acid sequence where at least one amino acid is modified.

Those of skilled in the art recognize that individual additions or substitutions to an amino acid sequence which alter a single amino acid or a small percentage of amino acids tend to result in the conservation of the properties of the original amino acid side-chain. As such, they are often referred to as “conservative substitutions” or “conservative modifications”, wherein the alteration of a protein results in a modified protein having a function analogous to the original protein. Conservative substitution tables providing functionally similar amino acids are well known in the art. Examples of properties of amino acid side chains are hydrophobic amino acids (A, I, L, M, F, P, W, Y, V), hydrophilic amino acids (R, D, N, C, E, Q, G, H, K, S, T), and side chains having the following functional groups or characteristics in common: an aliphatic side-chain (G, A, V, L, I, P); a hydroxyl group containing side-chain (S, T, Y); a sulfur atom containing side-chain (C, M); a carboxylic acid and amide containing side-chain (D, N, E, Q); a base containing side-chain (R, K, H); and an aromatic containing side-chain (H, F, Y, W).

In some embodiments, the artificial peptide is a peptide synthetically prepared based on a design obtained in silico using computer-based computational approaches.

In some embodiments, there is provided an artificial peptide comprises the amino acid sequence set forth in SEQ ID NO: 1.

In some embodiments, the artificial peptide is consisting of the amino acid sequence set forth in SEQ ID NO: 1.

In some embodiments, there is provided a conjugate comprising the artificial peptide of SEQ ID NO: 1 and a moiety linked thereto, optionally via a spacer, wherein the moiety is selected from the group consisting of a fluorescent probe, a photosensitizer, a chelating agent and a therapeutic agent.

The moiety of the conjugate as aforementioned may exhibit at least one of the following characteristics: (a) increased stability of hGBE1 protein; (b) enhanced transport into cells of the artificial peptide; (c) reduced half maximal inhibitory concentration (IC₅₀) of the artificial peptide in cytotoxicity; (d) enhanced efficacy of the artificial peptide in vivo; and (f) prolong an overall survival rate in a subject having a glycogen storage disorder.

In some embodiments, the moiety may be linked to the artificial peptide at the C-terminus thereof.

In some embodiments, the moiety may be linked to the artificial peptide at the N-terminus thereof.

In some embodiments, the moiety may be linked to the artificial peptide at both ends of the peptide.

In some embodiments, the moiety may be directly linked to the artificial peptide.

In some embodiments, the moiety may be optionally linked to the peptide via a spacer.

The term “spacer” as used herein is interchangeable with the terms “spacer moiety” and “spacer group” and refers to a component connecting the artificial peptide to the moiety thereby form a conjugate. Non-limiting examples of spacers include one or more natural or non-natural amino acids, a short peptide having no more than 8 amino acids and a C₁-C₂₅ alkyl.

The term “alkyl” as used herein refers to a fully saturated monovalent radical containing carbon and hydrogen, and which may be cyclic, branched or a straight chain. Non-limiting examples of alkyl groups are methyl, ethyl, n-butyl, n-hexyl, n-heptyl, n-octyl, n-nonyl, n-decyl, isopropyl, 2-methylpropyl, cyclopropyl, cyclopropylmethyl, cyclobutyl, cyclopentyl, cyclopen-tylethyl, cyclohexylethyl, cyclohexyl, cycloheptyl.

In some embodiments, the moiety may be a fluorescent probe.

In some embodiments, the fluorescent probe may be BPheide taurine amide (BTA), fluorenyl isothiocyanate (FITC), dansyl, rhodamine, eosin or erythrosine.

In some embodiments, the moiety if FITC.

In some embodiments, there is provided a pharmaceutical composition comprising the artificial peptide disclosed herein and a pharmaceutically acceptable carrier.

As used herein the term “pharmaceutical composition” or “composition” means one or more active ingredients, such as, the artificial peptide or a conjugate comprising same, and one or more inert ingredients, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention may encompass any composition made by admixing a compound of the present invention and a pharmaceutically acceptable excipient (pharmaceutically acceptable carrier).

In some embodiments, there is provided a pharmaceutical composition comprising the conjugate disclosed herein and a pharmaceutically acceptable carrier.

In some embodiments, there is provided use of the pharmaceutical compositions disclosed herein for the treatment of a disease or disorder associated with glycogen storage.

The term “treating” and “treatment” as used herein are interchangeable and refer to abrogating, inhibiting, slowing or reversing the progression of a disease or condition associated with glycogen storage, ameliorating clinical symptoms of a disease or condition or preventing the appearance or progression of clinical symptoms of a disease or condition associated with glycogen storage.

In some embodiments, a pharmaceutical effective amount of the pharmaceutical composition is used. The term “effective” is used herein, unless otherwise indicated, to describe an amount of the artificial peptide, the conjugate or composition comprising same which, in context, is used to produce or effect an intended result (e.g. the treatment of a disease or disorder associated with glycogen storage). The term effective subsumes all other effective amount or effective concentration terms which are otherwise described or used in the present application.

In some embodiments, the disease or disorder associated with glycogen storage is any one or more of glycogen storage disorder type IV (GSDIV) and late-onset adult polyglucosan body disease (APBD).

In some embodiments, there is provided a method of treating disease or disorder associated with glycogen storage in a subject in need thereof, the method comprising administering to said subject a pharmaceutical composition comprising an artificial peptide comprising the amino acid sequence set forth in SEQ ID NO: 1

The terms “subject” or “patient” are used throughout the specification within context to describe an animal, preferably a human, to whom a treatment or procedure, including a prophylactic treatment or procedure is performed.

The compositions of the present invention may be administered orally, parenterally, by inhalation spray, topically, transdermally, rectally, nasally, buccally, vaginally or via an implanted reservoir. The term “parenteral” as used herein includes subcutaneous, intravenous, intramuscular, intra-articular, intra-synovial, intrastemal, intrathecal, intrahepatic, intralesional and intracranial injection or infusion techniques. Preferably, the compositions are administered orally, intraperitoneally, or intravenously.

In some embodiments, the compositions of the invention will be administered intravenously for a period of at least one week. In some embodiments, the compositions of the invention will be administered intravenously for a period of at least two weeks. In some embodiments, the compositions of the invention will be administered intravenously for a period of at least 3 weeks. In some embodiments, the compositions of the invention will be administered intravenously for a period of about a month.

In some embodiment, the composition is administered by a first route of administration for a first period following administration by a second route of administration for a second period.

In some embodiment, the composition is administered intravenously for a first period following administration subcutaneously or intraperitonealy (IP) for a second period.

Sterile injectable forms of the compositions of the invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils conventionally employed as a solvent or suspending medium may be included. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of the invention may be orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include, but are not limited to, cocoa butter, beeswax and polyethylene glycols.

The pharmaceutical compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation or in a suitable enema formulation. Topically administered transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted, sterile saline, or as solutions in isotonic, pH adjusted, sterile saline, with or without a preservative such as benzylalkonium chloride. Alternatively, for ophthalmic uses, the pharmaceutical compositions may be formulated in an ointment.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of compound of the instant invention that may be combined with the carrier materials to produce a single dosage form will vary depending upon the host treated, the type and/or stage of the disease and the particular mode of administration.

It should also be understood that a specific dosage and treatment regimen for any particular patient will depend upon a variety of factors, including the activity of the specific compound employed, the age, body weight, general health, gender, diet, time of administration, rate of excretion, drug combination, and the judgment of the treating physician and the severity of the particular disease or condition being treated.

Administration of the active compound may range from continuous (intravenous drip) to several oral administrations per day (for example, four times a day (Q.I.D.)) and may include oral, topical, parenteral, intramuscular, intravenous, sub-cutaneous, transdermal (which may include a penetration enhancement agent), buccal and suppository administration, among other routes of administration. Enteric coated oral tablets may also be used to enhance bioavailability of the compounds from an oral route of administration. The most effective dosage form will depend upon the pharmacokinetics of the particular agent chosen as well as the severity of disease in the patient. Oral dosage forms are preferred, because of ease of administration and prospective favorable patient compliance.

To prepare the pharmaceutical compositions according to the present invention, a therapeutically effective amount of one or more of the compounds according to the present invention is preferably intimately admixed with a pharmaceutically acceptable carrier according to conventional pharmaceutical compounding techniques to produce a dose. A carrier may take a wide variety of forms depending on the form of preparation desired for administration. In preparing pharmaceutical compositions in oral dosage form, any of the usual pharmaceutical media may be used. Thus, for liquid oral preparations such as suspensions, elixirs and solutions, suitable carriers and additives including water, glycols, oils, alcohols, flavouring agents, preservatives, colouring agents and the like may be used. For solid oral preparations such as powders, tablets, capsules, and for solid preparations such as suppositories, suitable carriers and additives including starches, sugar carriers, such as dextrose, mannitol, lactose and related carriers, diluents, granulating agents, lubricants, binders, disintegrating agents and the like may be used. If desired, the tablets or capsules may be enteric-coated or sustained release by standard techniques. The use of these dosage forms may significantly the bioavailability of the compounds in the patient.

For parenteral formulations, the carrier will usually comprise sterile water or aqueous sodium chloride solution, though other ingredients, including those which aid dispersion, also may be included. Of course, where sterile water is to be used and maintained as sterile, the compositions and carriers must also be sterilized. Injectable suspensions may also be prepared, in which case appropriate liquid carriers, suspending agents and the like may be employed.

Liposomal suspensions may also be prepared by conventional methods to produce pharmaceutically acceptable carriers.

In addition, compounds according to the present invention may be administered alone or in combination with other agents, including other compounds of the present invention. Certain compounds according to the present invention may be effective for enhancing the biological activity of certain agents according to the present invention by reducing the metabolism, catabolism or inactivation of other compounds and as such, are co-administered for this intended effect.

The invention is illustrated further in the following non-limiting examples.

### EXAMPLES

### Example 1—Recombinant hGBE1 Production, Crystallization and Characterization

DNA fragment encoding aa 38-700 of human GBE1 (hGBE1_{trunc}) was amplified from a cDNA clone (IMAGE: 4574938) and subcloned into the pFB-LIC-Bse vector (Gene Bank accession number EF199842) in frame with an N-terminal His₆-tag and a TEV protease cleavage site. Full-length hGBE1 was constructed in the pFastBac-1 vector, from which the hGBE1-Y329S mutant was generated by two sequential PCR reactions. hGBE1 protein was expressed in insect cells in Sf9 media and purified by affinity and size-exclusion chromatography. hGBE1 was crystallized by vapor diffusion at 4° C. Diffraction data were collected at the Diamond Light Source. Phases for hGBE1 were calculated by molecular replacement.

### Example 2—hGBE1 Structure Determination

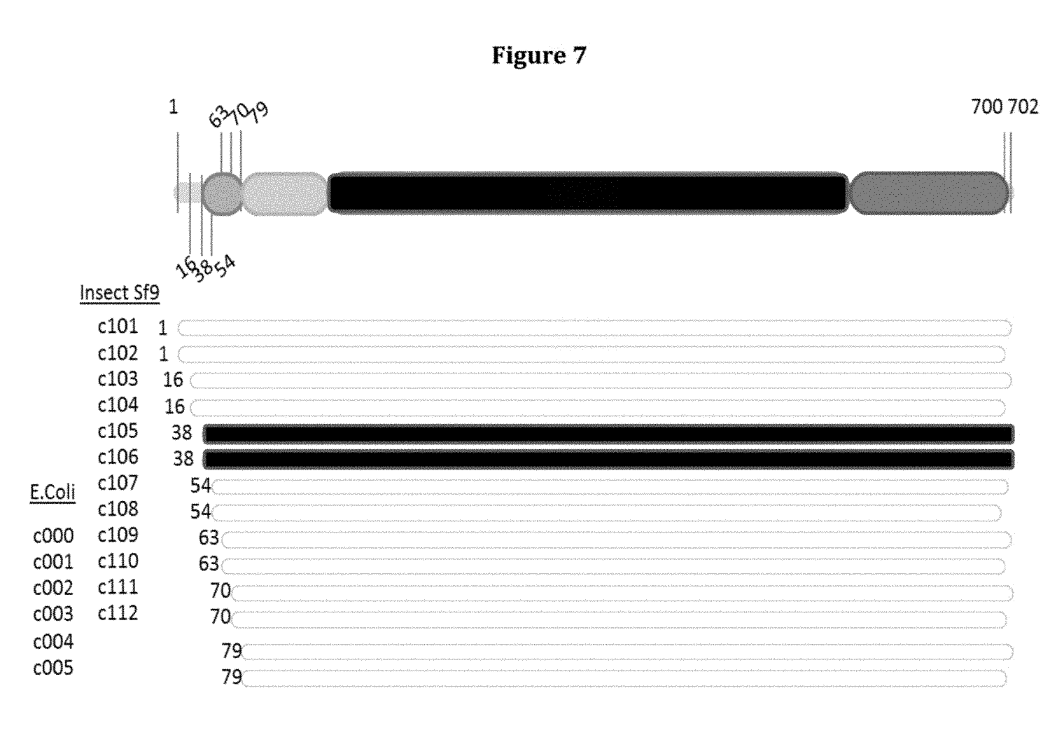

Baculovirus-infected insect cell overexpression of hGBE1, a 702-amino acid (aa) protein were used for structural studies. Interrogation of several N- and C-terminal boundaries (FIG. 7) in this expression system yielded a soluble and crystallisable polypeptide for hGBE1 from amino acids (aa) 38-700 (hGBE1trunc). Using the molecular replacement method with the Oryza sativa starch branching enzyme I (SBE1; PDB: 3AMK; 54% identity to hGBE1) as search model, the following structures were determined: the structure of hGBE1trunc in the apo form (hGBE1-apo) and in complex with the tetrasaccharide acarbose (hGBE1-ACR) or heptasaccharide maltoheptaose (hGBE1-Glc7) to the resolution range of 2.7-2.8 Å (Table 1). Inspection of the asymmetric unit content as well as symmetry-related protomers did not reveal any stable oligomer arrangements, consistent with GBE1 being a monomer in size exclusion chromatography, similar to most GH13 enzymes.

TABLE 1
Crystallography refinement statistics.

| | hGBE1-apo | | hGBE1-ACR | | hGBE1-Glc7 | |
|---|---|---|---|---|---|---|
| Overall Description | | | | | | |
| Pdb code | 4BZY | | xxxx | | xxxx | |
| Ligands bound | — | | ACR | | Glc7 | |
| Data collection | | | | | | |
| Beamline | Diamond I04-1 | | Diamond I03 | | Diamond I04 | |
| Wavelength (Å) | 0.92 | | 0.9795 | | 0.9795 | |
| Unit cell parameters (Å) | 117.3, 164.5, | | 116.8, 164.0, | | 116.7, 164.5, | |
| | 311.3 | | 311.7 | | 313.2 | |
| α = β = γ(°) | 90 | | 90 | | 90 | |
| Space group | C222₁ | | C222₁ | | C222₁ | |
| Resolution range (Å) | 91.3-2.75 | | 72.5-2.79 | | 313-2.80 | |
| | (2.90-2.75) | | (2.86-2.79) | | (3.13-2.80) | |
| Rmerge(%) | 0.174 | (0.873) | 0.118 | (0.697) | 0.153 | (0.758) |
| I/sig(I) | 16.3 | (2.0) | 10.0 | (2.1) | 9.2 | (2.1) |
| Completeness | 99.9 | (100.0) | 99.7 | (99.7) | 99.8 | (99.8) |
| Multiplicity | 18.1 | (7.8) | 3.8 | (3.8) | 4.5 | (4.7) |
| Refinement | | | | | | |

| Rcryst (%) | 0.1845 | 0.1937 | 0.1828 |
|---|---|---|---|
| Rfree (%) | 0.2251 | 0.2393 | 0.2152 |
| Wilson B factor (Å²) | 48.75 | 48.86 | 52.34 |
| Average total B factor (Å²) | 46.02 | 38.95 | 55.37 |
| Average ligand B factor (Å²) | n/a | 50.93 | 68.90 |
| Ligand occupancy | n/a | 1.00 | 1.00 |
| Rmsd bond length (Å) | 0.003 | 0.009 | 0.004 |
| Rmsd bond angle (°) | 0.759 | 1.253 | 0.81 |
| Ramachandran outliers (%) | 0.05 | 0.16 | 0.00 |
| Ramachandran favoured (%) | 98.06 | 97.64 | 97.96 |

### Data for Highest Resolution Shell are Shown in Parenthesis.

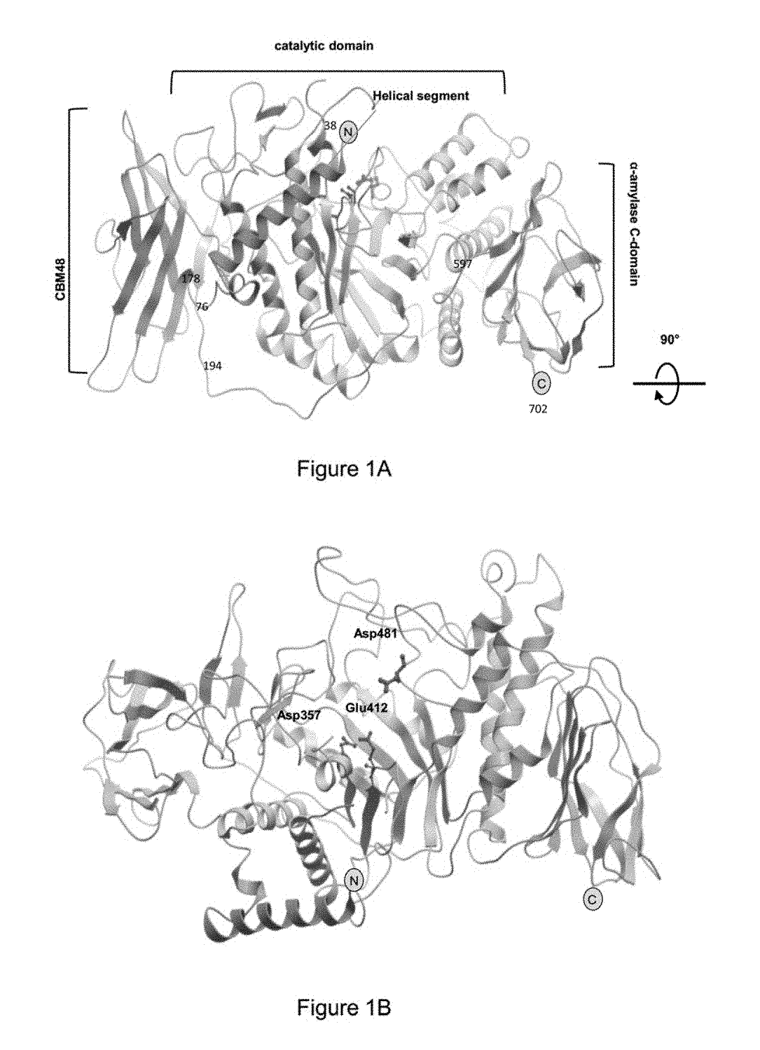

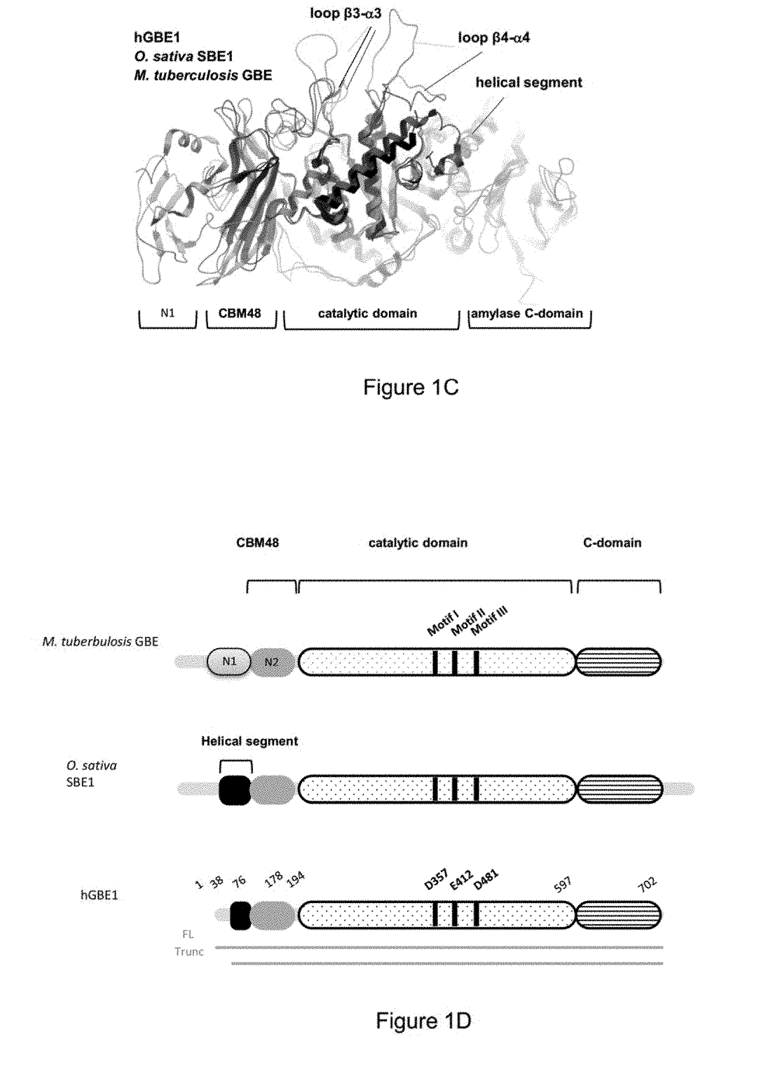

hGBE1 structure was found to have an elongated shape (longest dimension>85 Å) composed of four structural regions (FIGS. 1A and 1B): the N-terminal helical segment (aa 43-75), a carbohydrate binding module 48 (CBM48; aa 76-183), a central catalytic core (aa 184-600) and the C-terminal amylase-like barrel domain (aa 601-702). A structural overlay of hGBE1 with reported branching enzyme structures from O. sativa SBE1 (PDB: 3AMK, Cα-rmsd: 1.4 Å, sequence identity: 54%) and M. tuberculosis GBE (3K1D, 2.1 Å, 28%; FIG. 1C) highlights the conserved catalytic core housing the active site within a canonical (βα)6-barrel. Nevertheless the different branching enzymes show greater structural variability in the N-terminal region preceding the catalytic core, as well as in two surface-exposed loops of the TIM-barrel (FIG. 1C). For example, in O. sativa SBE1 and human GBE1 structures the helical segment precedes the CBM48 module, while in M. tuberculosis GBE the helical segment is replaced by an additional (3-sandwich module (denoted N1 in FIGS. 1C and 1D). The closer homology of hGBE1 with O. sativa SBE1, whose substrate is starch, than with the bacterial paralog M. tuberculosis GBE, suggests a similar evolutionary conservation in the branching enzyme mechanism for glycogen and starch, both involving a growing linear α1,4-linked glucan chain as substrate.

### Example 3—Oligosaccharide Binding of hGBE1 at Catalytic and Non-Catalytic Sites

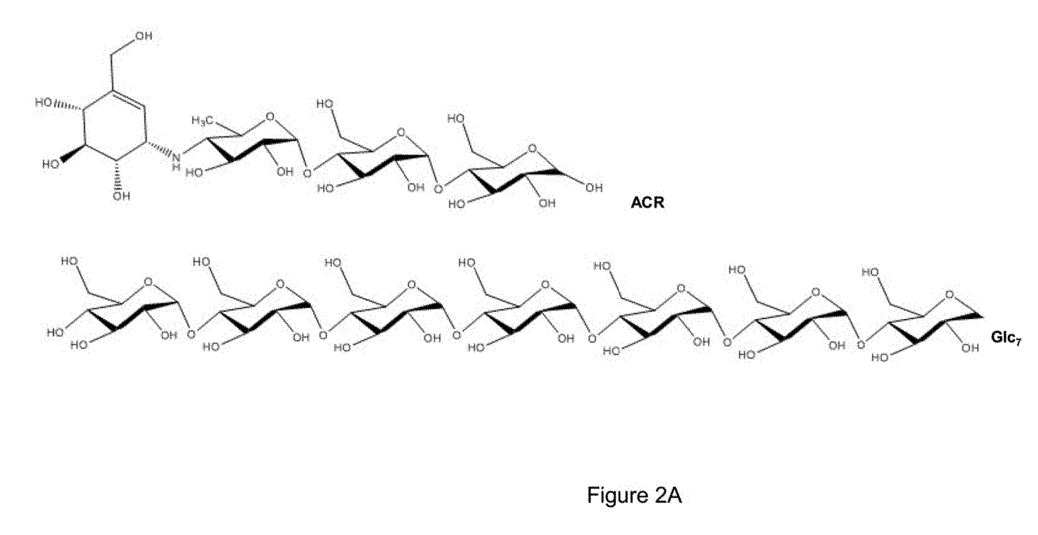

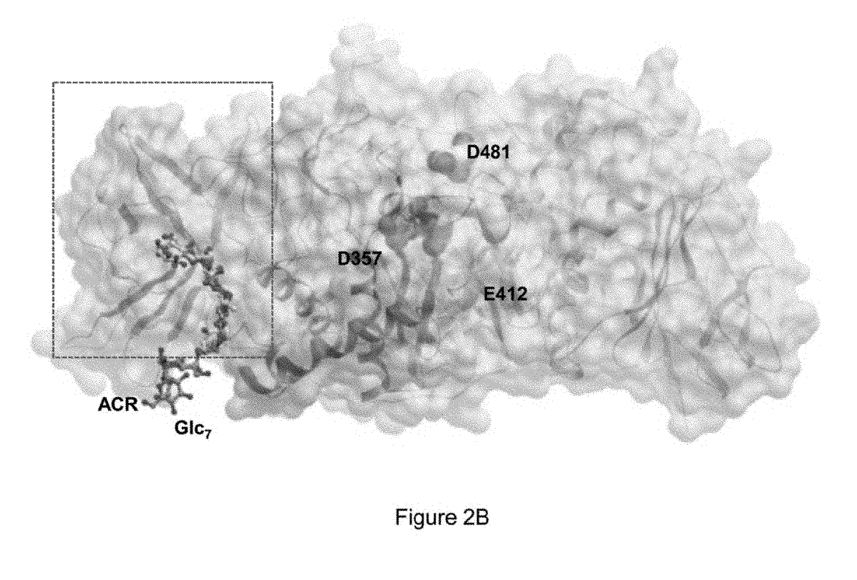

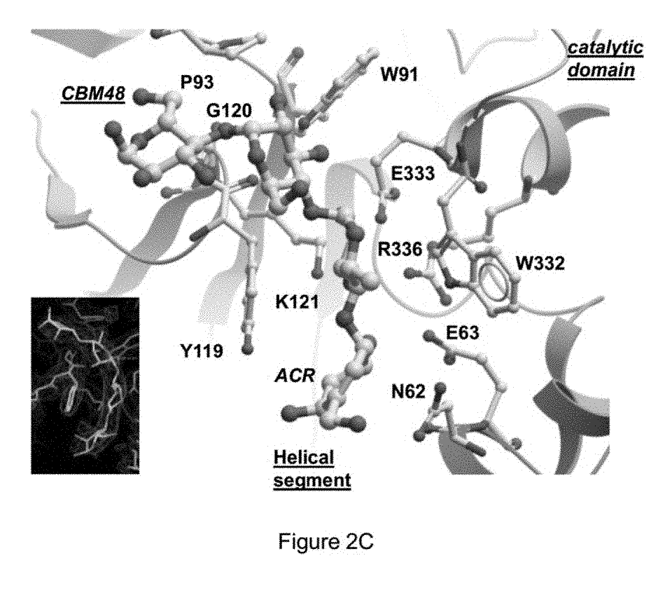

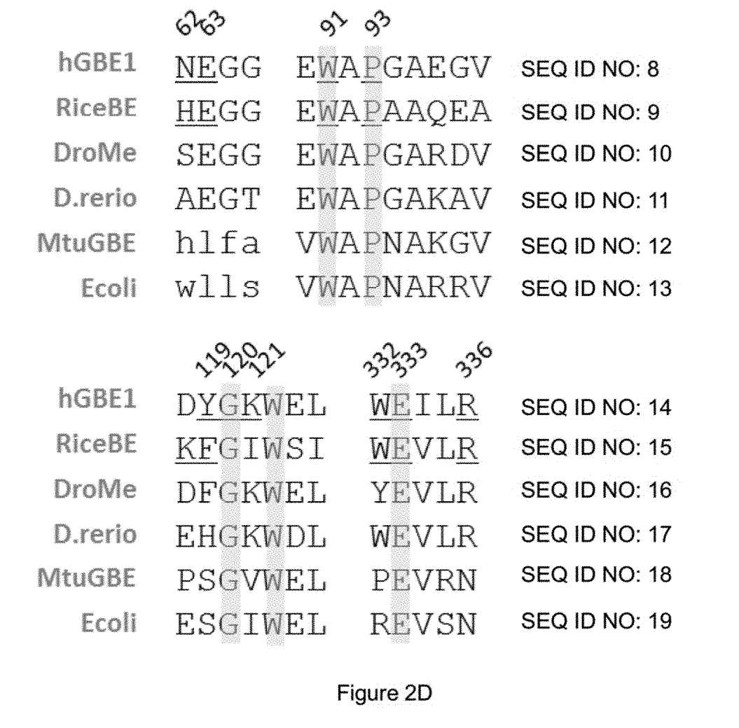

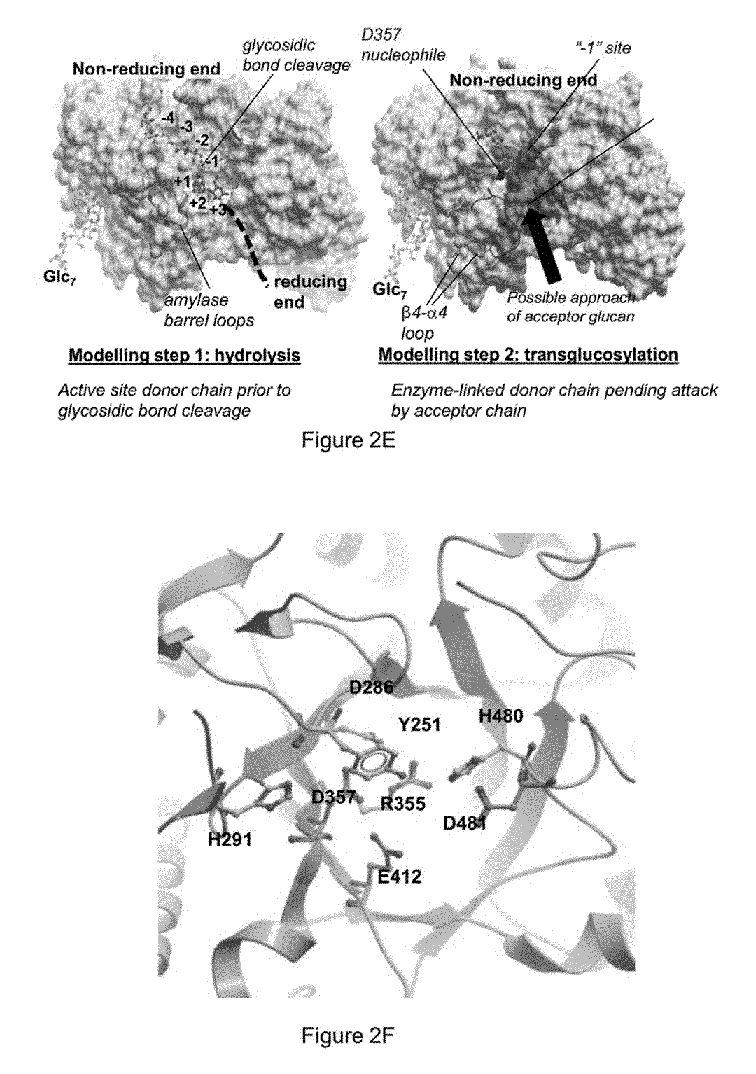

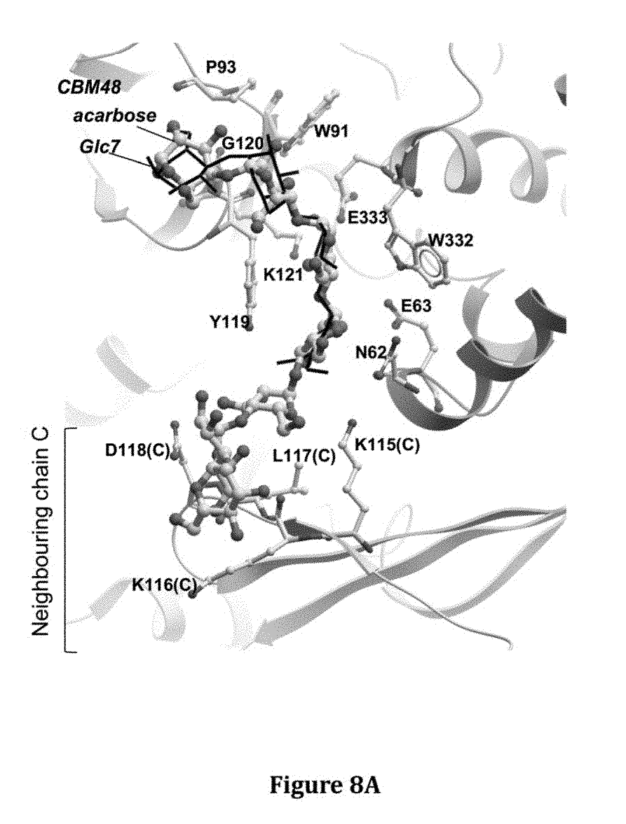

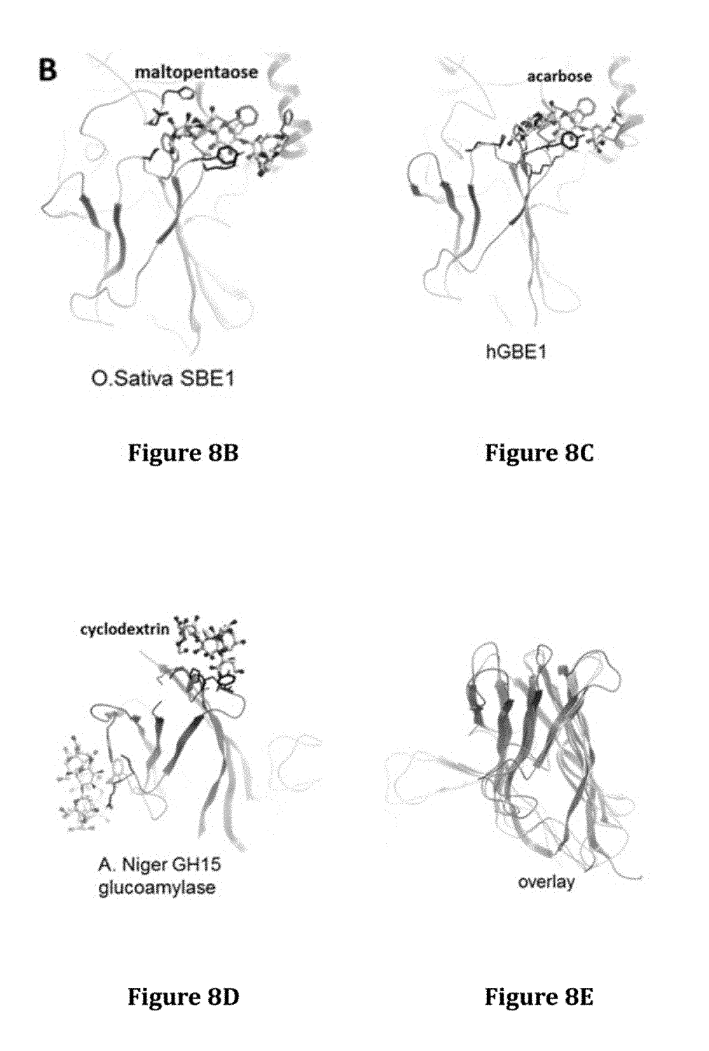

Co-crystallized hGBE1_{trunc} with acarbose (ACR) or maltoheptaose (Glc7) were used to characterize the binding of oligosaccharides to branching enzymes, (FIG. 2A). ACR is a pseudo-tetrasaccharide acting as active site inhibitor for certain GH13 amylases. In the hGBE1-ACR structure, acarbose is bound not at the expected active site, but instead at the interface between the CBM48 and the catalytic domains (FIG. 2B). Within this oligosaccharide binding cleft (FIG. 2C), ACR interacts with protein residues from the N-terminal helical segment (Asn62, Glu63), CBM48 domain (Trp91, Pro93, Tyr119, Glyl20, Lys121) as well as catalytic core (Trp332, Glu333, Arg336). These interactions, likely to be conserved among species (FIG. 2D), include hydrogen bonds to the sugar hydroxyl groups as well as hydrophobic/aromatic interactions with the pyranose rings. The hGBE1-Glc7 structure reveals similar conformation and binding interactions of maltoheptaose for its first four 1,4-linked glucose units (FIG. 2B). The three following glucose units, however, extend away from the protomer surface and engage in interactions with a neighboring non-crystallographic symmetry (NCS)-related protomer in the asymmetric unit (FIG. 8A). These artefactual interactions mediated by crystal packing are unlikely to be physiologically relevant.

CBM48 is a (3-sandwich module found in several GH13 amylolytic enzymes. The acarbose binding cleft observed here is the same location that binds maltopentaose in the O. sativa SBE1 structure, as well as other oligosaccharides in CBM48-containing proteins (FIG. 8B). The conserved nature of this non-catalytic cleft among branching enzymes (FIG. 2D), and its presumed higher affinity for oligosaccharides than the active site, may represent one of the multiple non-catalytic binding sites on the enzyme surface. They may provide GBEs the capability to anchor a complex glycogen granule and determine the chain length specificity for the branching reaction as a ‘molecular ruler’. This agrees with the emerging concept of glycogen serving not only as the substrate and product of its metabolism, but also as a scaffold for all acting enzymes.

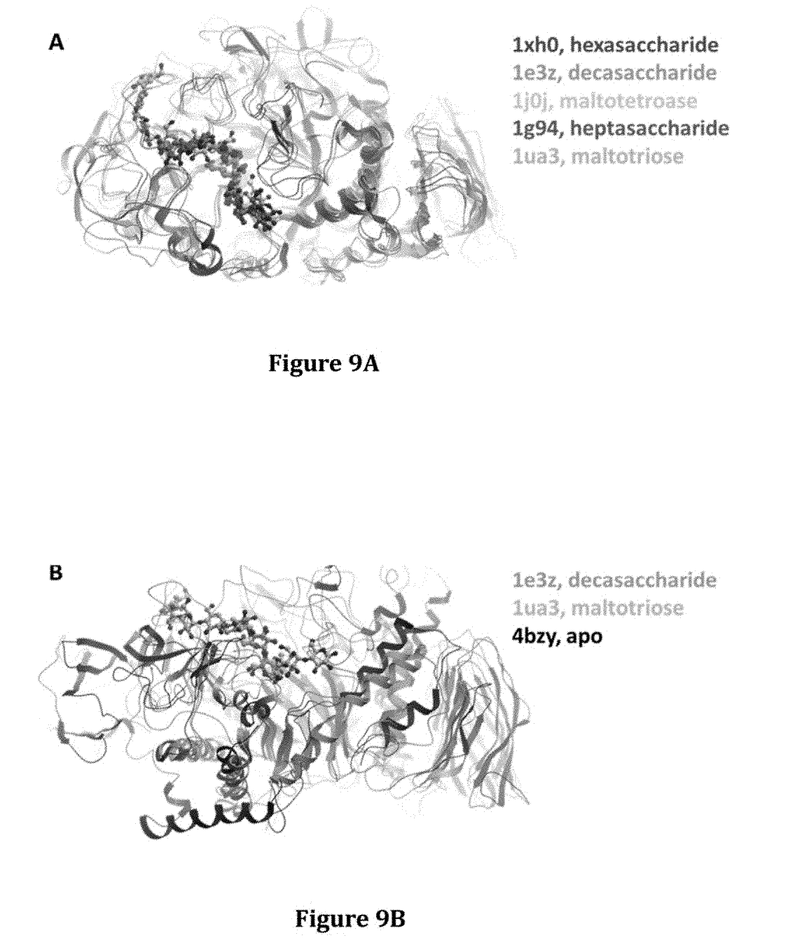

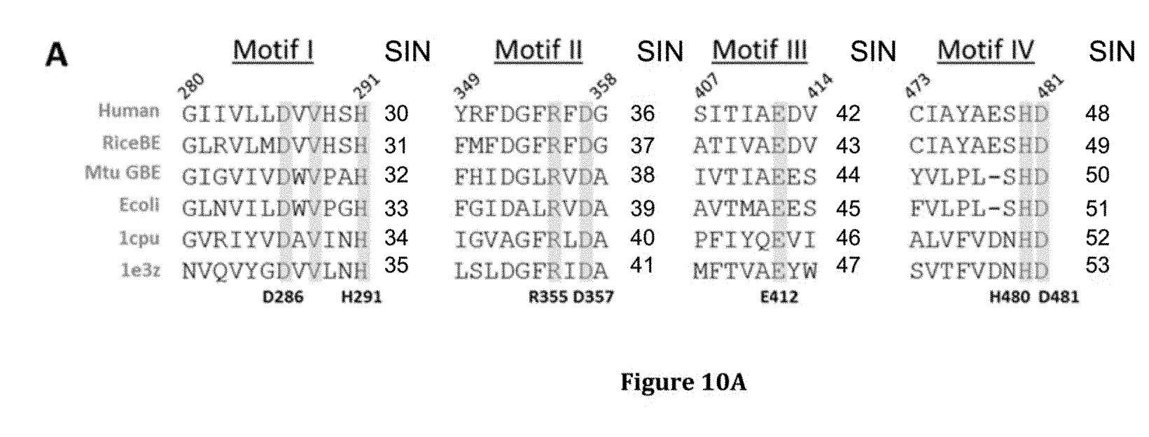

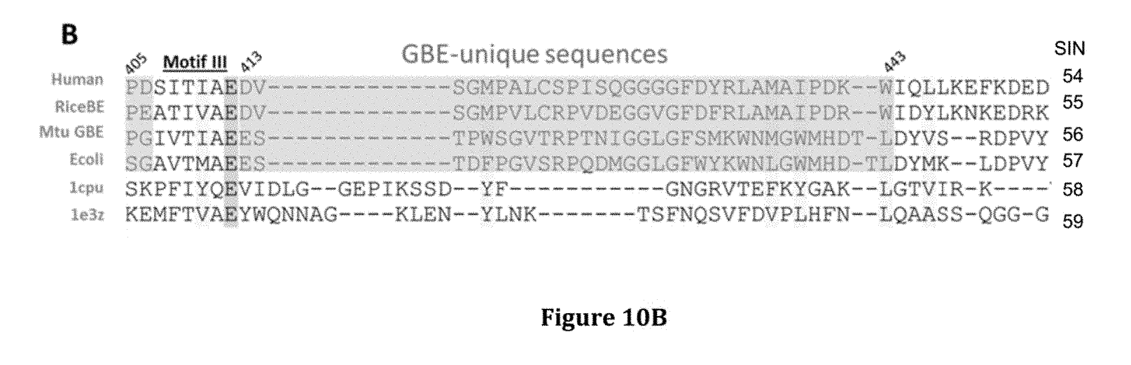

In light of the unsuccessful co-crystallization of hGBE1 with an active site-bound oligosaccharide, the analysis of the active site is guided by reported structures of GH13 α-amylases in complex with various oligosaccharides (FIG. 9A). The catalytic domain TIM-barrel of hGBE1 superimposes well with those from the amylase structures (RMSD 1.2 Å for 130-150 C^{α} atoms; FIG. 9B), suggesting a similar mode of substrate threading along the GH13 enzyme active sites, at least within the proximity of glycosidic bond cleavage. The hGBE1 active site is a prominent surface groove at the ((3a)₆-barrel that could bind a linear glucan chain via a number of subsites (FIG. 2E, left), each binding a single glucose unit. The subsites are named “−n”, . . . “−1”, “+1”, “+n”, denoting the n^{th} glucose unit in both directions from the scissile glycosidic bond. The most conserved among GH13 enzymes is the “−1” subsite, which harbors seven strictly conserved residues forming the catalytic machinery (FIGS. 2F and 10A). The other subsites lack a significant degree of sequence conservation, suggesting that substrate recognition other than at the “−1” subsite is mediated by surface topology and shape complementarity, and not sequence-specific interactions.

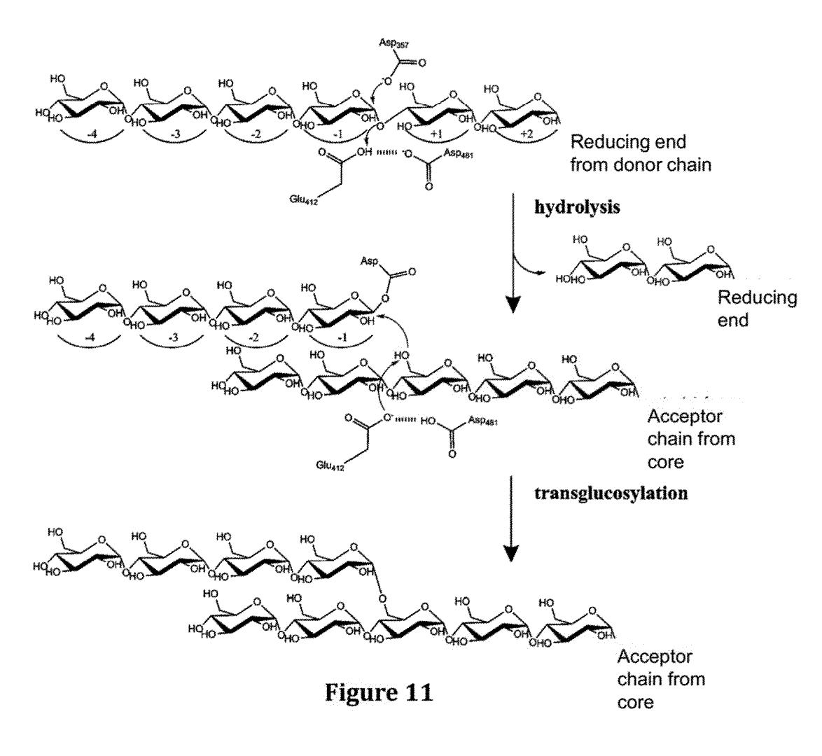

The task of the hGBE1 active site is to catalyze two reaction steps (hydrolysis and transglucosylation) on a growing glucan chain (FIG. 11). The first reaction is a nucleophilic attack on the “−1” glucose at the C-1 position by an aspartate (Asp357), generating a covalent enzyme-glycosyl intermediate with release of the remainder of the glucan chain carrying the reducing end (+1, +2 . . . ). In the second reaction, the enzyme-linked “−1” glucose is attacked by a glucose 6-hydroxyl group from either the same or another glucan chain, which acts as a nucleophile for the chain transfer. While both hGBE1 reactions presumably proceed via a double displacement mechanism involving the strictly conserved triad Asp357-Glu412-Asp481, as proposed for GH13 amylases, there exist mechanistic differences between branching and amylolytic enzymes: (i) the branching enzyme substrate is not a malto-oligosaccharide, but rather a complex glycogen granule with many glucan chains; (ii) the transglycosylation step in GBE (glucose 6-OH as acceptor) is replaced by hydrolysis in amylases (H₂O as acceptor). These differences require that the active site entrance of hGBE1 be tailor-made to accommodate the larger more complex glucose acceptor chain (FIG. 2E), as opposed to a water molecule in amylases. A region of GBE-unique sequences (aa 405-443), rich in Gly/Ala residues, has been identified based on alignment with GH13 sequences (FIG. 10B). This region, replaced in amylolytic enzymes by sequence insertions and bulkier residues, maps onto a hGBE1 surface that is proximal to the “+1, +2 . . . ” subsites, and to the β4-α4 loop that is disordered in hGBE1 but adopts different conformations in the O. sativa and M. tuberculosis structures (FIG. 1B and FIG. 2E, right). This surface region, unique to branching enzymes, may facilitate access to the active site by an incoming glucan acceptor chain.

### Example 4—GBE1 Missense Mutations in the Catalytic Core

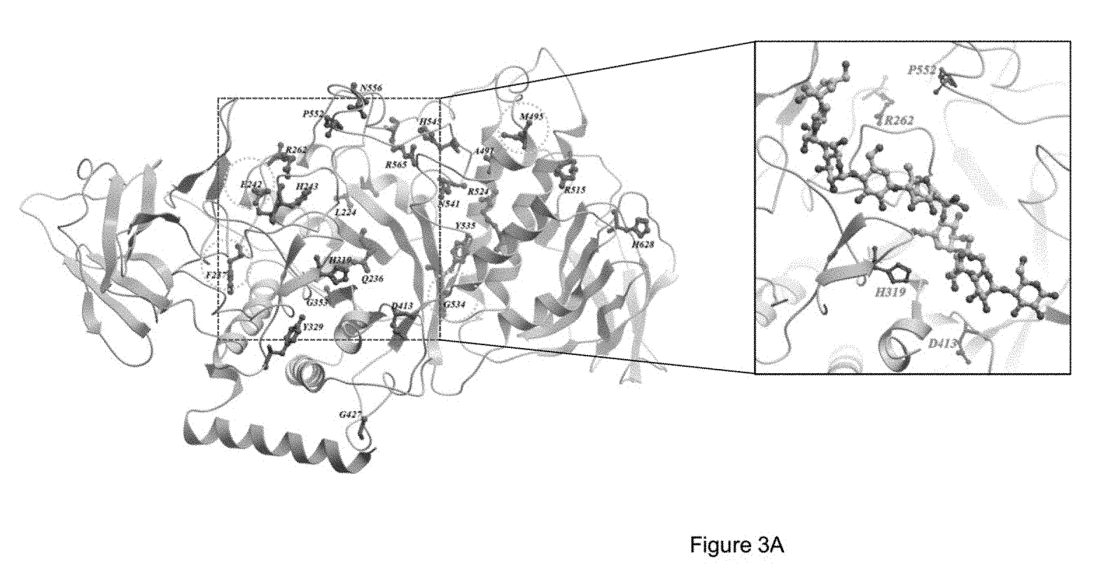

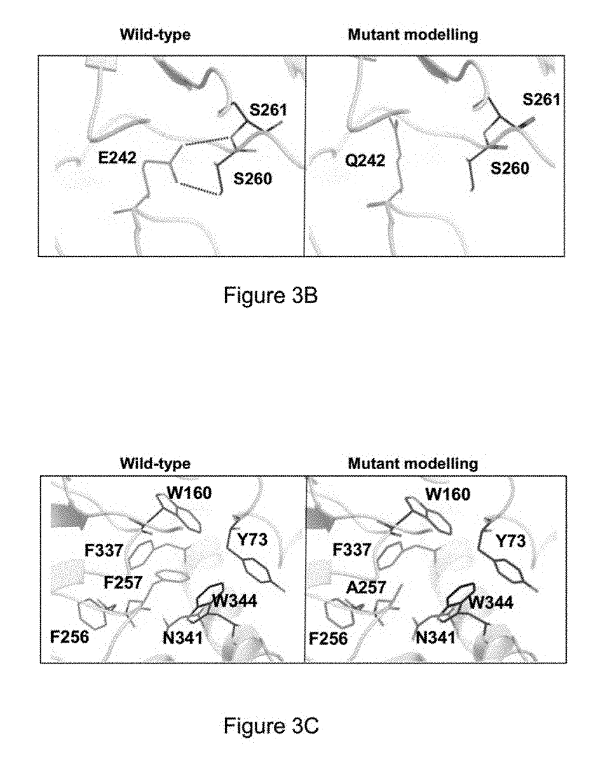

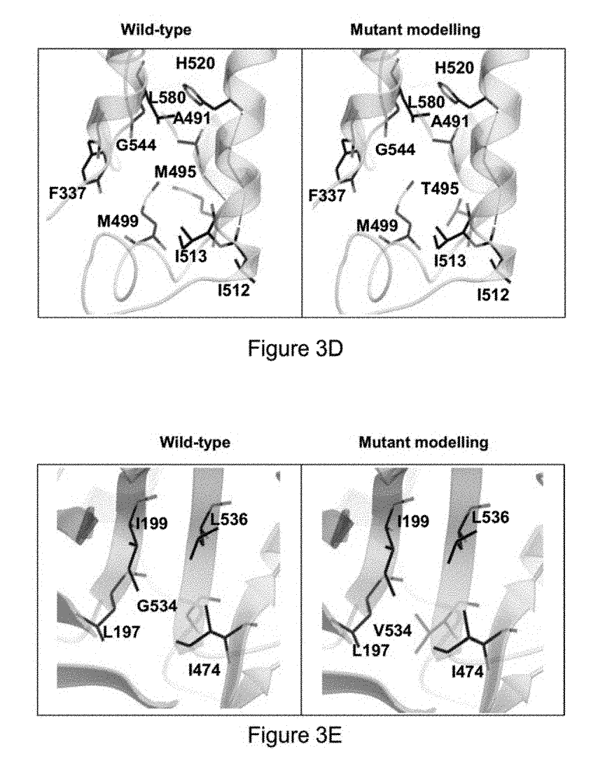

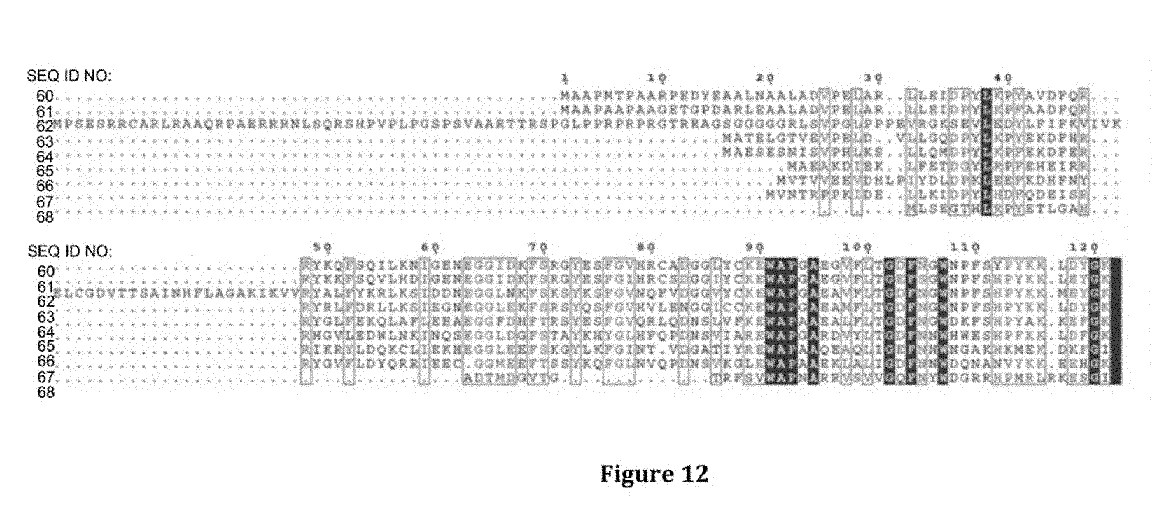

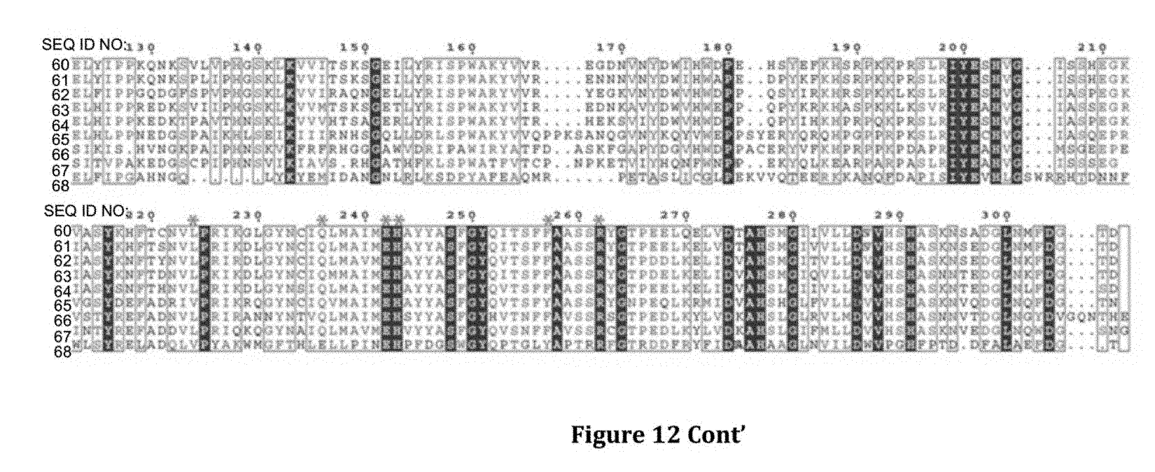

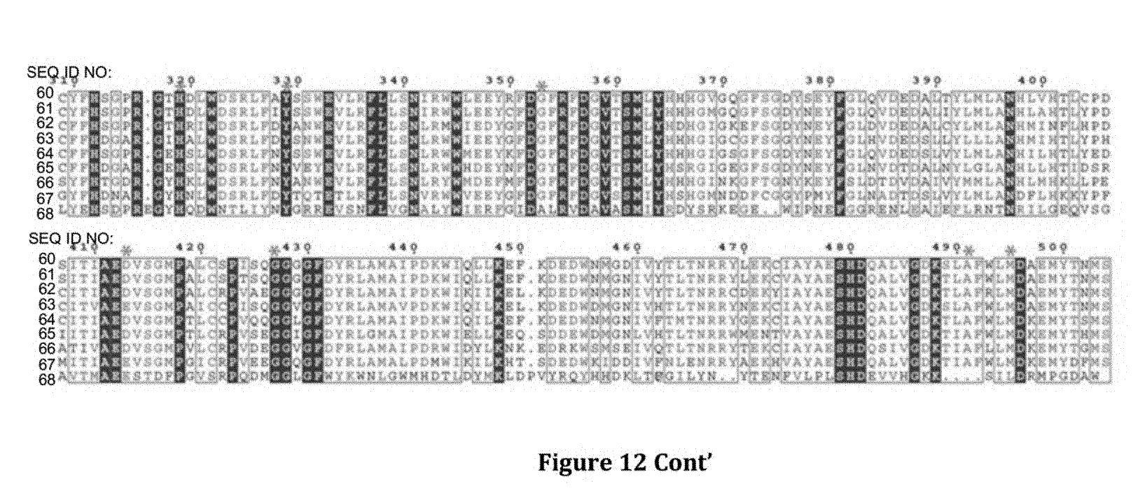

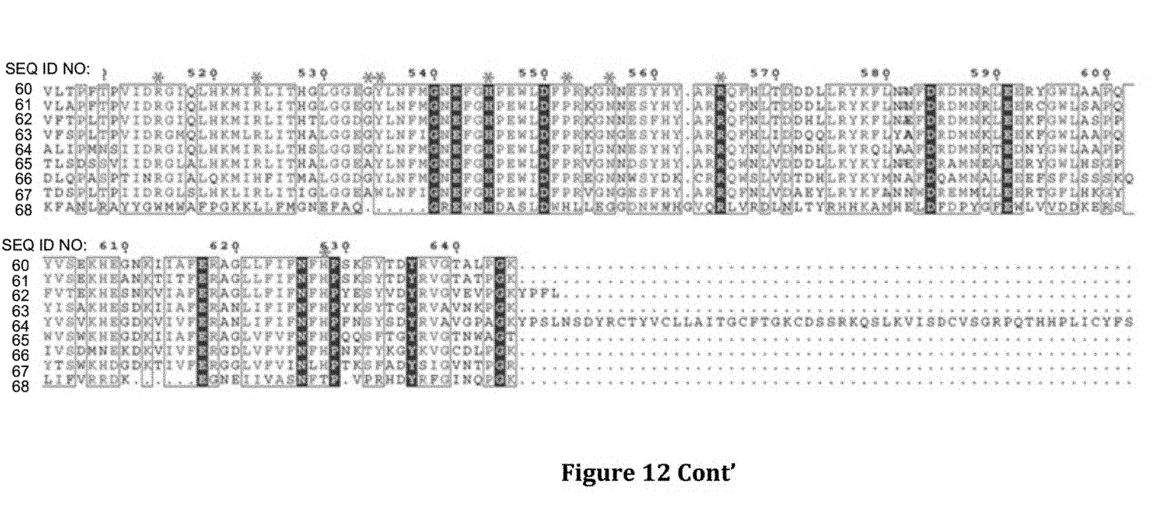

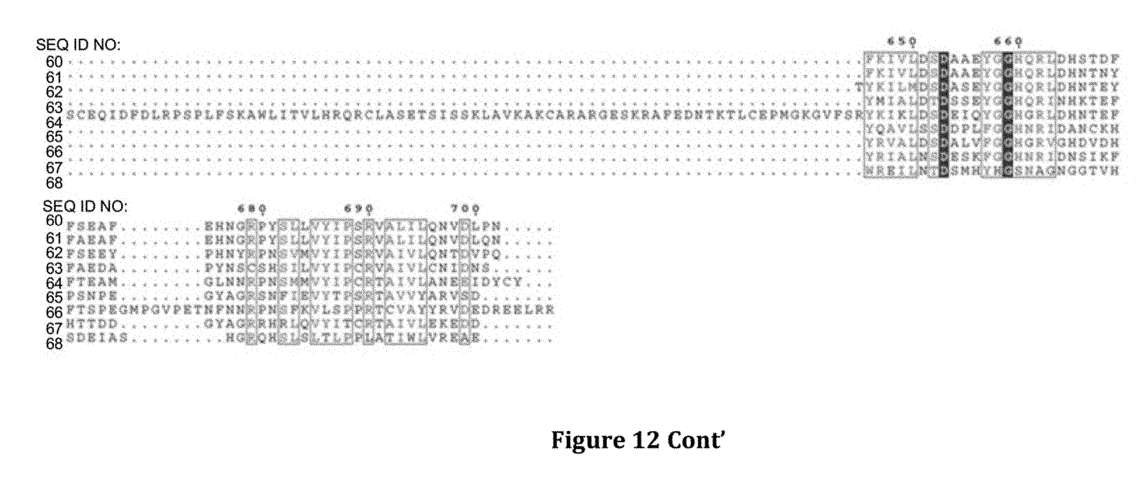

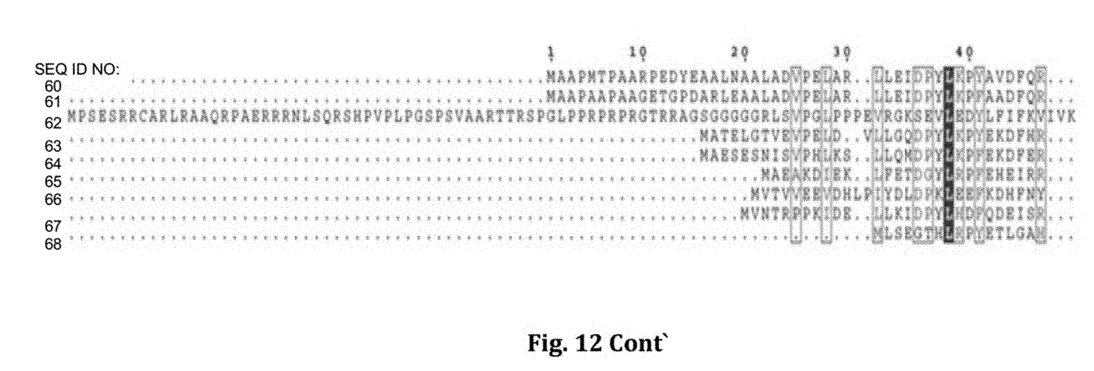

The hGBE1 crystal structure provides a molecular framework to understand the pathogenic mutations causing GSDIV and APBD, as the previously determined bacterial GBE structures have low amino acid conservation in some of the mutated positions. Apart from a few large-scale aberrations (nonsense, frameshift, indels, intronic mutations), which likely result in truncated and non-functional enzyme, there are to date 25 reported GBE1 missense mutations, effecting single amino acid changes at 22 different residues (Table 2). These mutation sites are predominantly localized in the catalytic core (FIG. 3A), with a high proportion around exon 12 (n=6 in exon 12, n=2 in exon 13, n=1 in exon 14). There is no apparent correlation among the genotype, amino acid change and its associated disease phenotype. However, inspection of the atomic environment surrounding these residues, some of which are strictly invariant among GBE orthologs (FIG. 12), allows us to postulate their molecular effects. They may be classified into ‘destabilising’ substitutions, which likely disrupt protein structure, and ‘catalytic’ substitutions, which are located proximal to the active site and may affect oligosaccharide binding or catalysis. The most common type of ‘destabilising’ mutations is those disrupting H-bond networks (p.Q236H, p.E242Q, p.H243R, p.H319R/Y, p.D413H, p.H545R, p.N556Y, p.H628R; FIG. 3B) and ionic interactions (p.R262C, p.R515C/H, p.R524Q, p.R565Q) within the protein core, while disruption of aromatic or hydrophobic interactions are also common (p.F257L, p.Y329S/C, p.Y535C, p.P552L; FIG. 3C). Also within the protein core, mutation of a large buried residue to a small one creates a thermodynamically un-favored cavity (p.M495T, p.Y329S/C; FIG. 3D), while mutation from a small residue to a bulkier one creates steric clashes with the surroundings (p.G353A, A491Y, p.G534V; FIG. 3E). In certain cases, mutation to a proline within an α-helix likely disrupts local secondary structure (e.g. p.L224P), while mutation from glycine can lose important backbone flexibility (e.g. p.G427R, likely causing Gln426 from the catalytic domain to clash with Phe45 in the helical segment). The ‘catalytic’ mutations are more difficult to define in the absence of a sugar bound hGBE1 structure at the active site. However, superimposing hGBE1 with amylase structures reveals Arg262, His319, Asp413 and Pro552 as mutation positions that could line the oligosaccharide access to the active site (FIG. 3A, inset). In particular, the imidazole side-chain of His319 is oriented towards the active site and within 8 Å distance from the −1 site. Its substitution to a charged (p.H319R) or bulky (p.H319Y) amino acid may destabilize oligosaccharide binding.

TABLE 2
List of GBE1 missense mutations

| Protein | DNA | | |
|---|---|---|---|
| Change | change | Exon | Disease phenotypes |
| p.L224P | c.671C > T | 5 | Nonprogressive hepatic; APBD |
| p.Q236H | c.708G > C | 6 | Childhood neuromuscular (mild) |
| p.E242Q | c.724G > C | 6 | APBD |
| p.H243R | c.728A > G | 6 | Neonatal neuromuscular |
| p.F257L | c.771T > A | 6 | Classic hepatic |
| p.R262C | c.784C > T | 7 | Childhood neuromuscular (mild) |
| p.H319R | c.956A > G | 7 | foetal akinesia deformation |
| p.H319Y | c.955C > T | 7 | APBD |
| p.Y329S | c.986A > C | 8 | Nonprogressive hepatic, APBD |
| p.Y329C | c.986A > G | 8 | APBD |
| p.G353A | c.1058G > C | 8 | APBD |
| p.D413H | c.1237G > C | 10 | APBD |
| p.G427R | c.1279G > A | 10 | Classic hepatic |
| p.A491Y | c.1471G > C | 12 | foetal akinesia deformation |
| p.M495T | c.1484T > C | 12 | Classic hepatic |
| p.R515C | c.1543C > T | 12 | Classic hepatic |
| p.R515H | c.1544G > A | 12 | APBD |
| p.R524Q | c.1571G > A | 12 | APBD, classic hepatic |
| p.G534V | c.1601G > T | 12 | APBD |
| p.Y535C | c.1604A > G | 12 | Classic hepatic |
| p.N541D | c.1623A > G | 13 | APBD |
| p.H545R | c.1634A > G | 13 | Neonatal neuromuscular |
| p.P552L | c.1655C > T | 13 | Classic hepatic |
| p.N556Y | c.1666A > T | 13 | APBD |
| p.R565Q | c.1694G > A | 13 | APBD |
| p.H628R | c.1883A > G | 14 | Childhood neuromuscular |

### Example 5—GBE1 p.Y329S: A Destabilizing Mutation

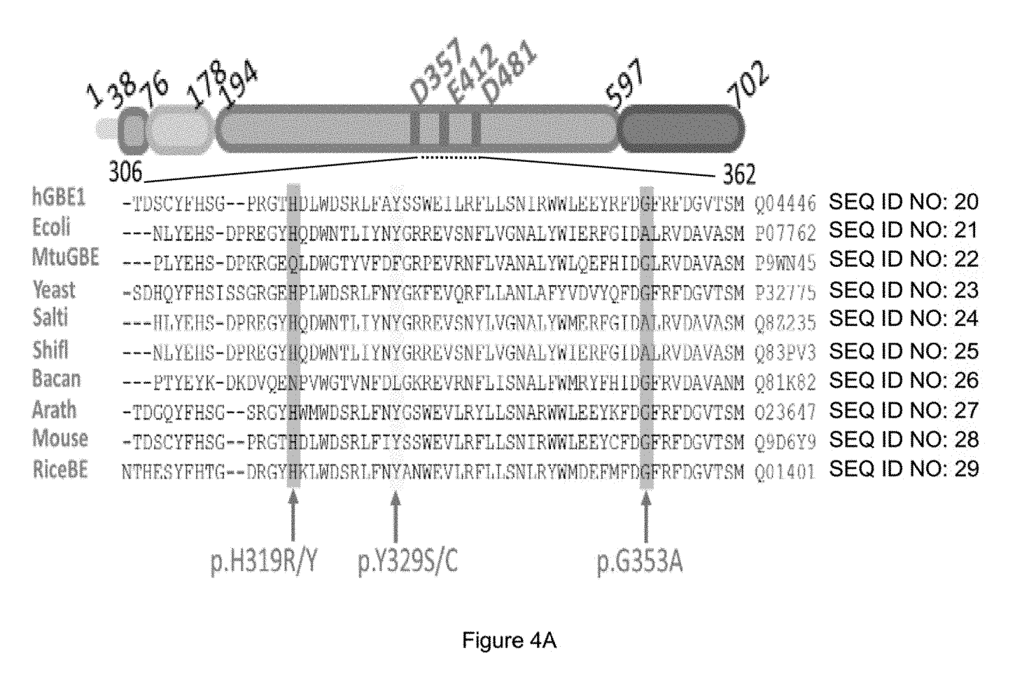

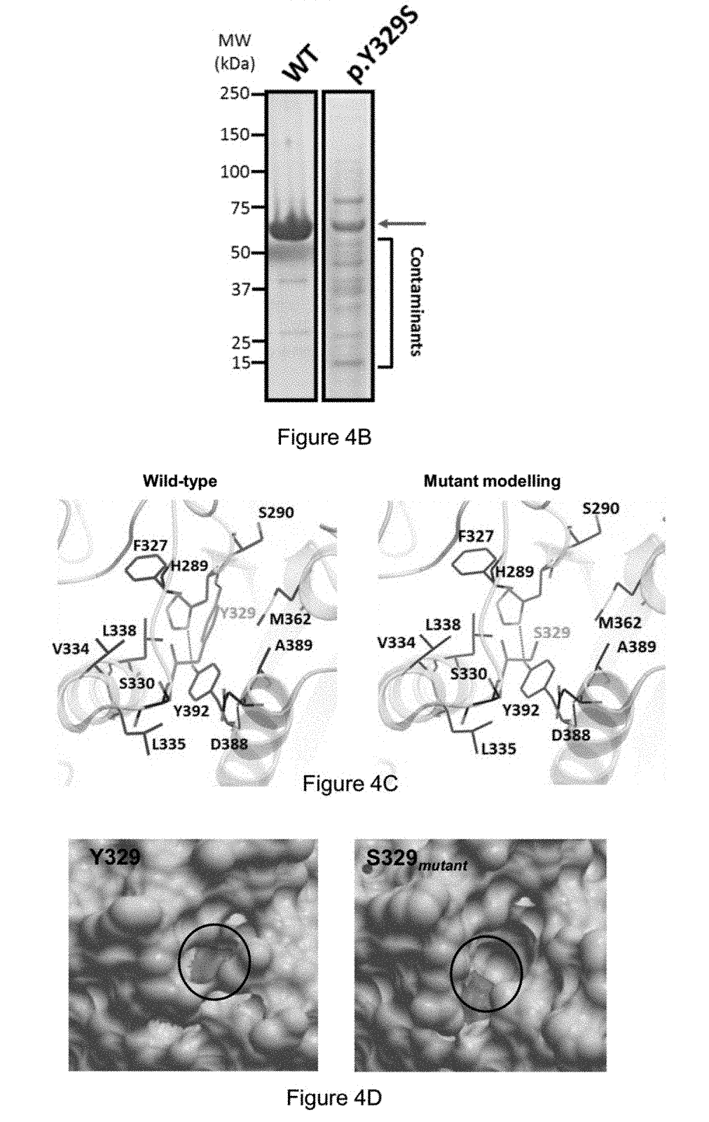

The c.986A>C mutation results in the p.Y329S amino acid substitution, the most common APBD-associated mutation. This residue is highly conserved across different GBE orthologs supporting its associated pathogenicity (FIG. 4A). Compared to wild type, a drastic reduction in recombinant expression and protein solubility from a hGBE1 construct harboring the p.Y329S substitution was observed (FIG. 4B). Based on the protein structure, it may be deduced that Tyr329 is a surface exposed residue in the catalytic domain, that confers stability to the local environment by interacting with the hydrophobic residues Phe327, Val334, Leu338, Met362 and Ala389. Mutation of Tyr329 to the smaller amino acid serine (Ser329ₘᵤₜₐₙₜ) likely removes these interactions (FIG. 4C, right) and creates a solvent accessible cavity within this hydrophobic core (FIG. 4D), which could lead to destabilized protein.

The aforementioned data indicate that the p.Y329S mutation, which is associated with APBD, results in protein destabilization.

### Example 6—Computational Design of hGBE1 p.Y329S-Stabilizing Peptide

To facilitate the design of a small molecule/peptide chaperone, which could confer stability to the Ser329ₘᵤₜₐₙₜ site, a structural model of hGBE1-Y329S was generated from the wild-type hGBE1-apo coordinates.

cDNA encoding full-length hGBE1 was produced by PCR using primers that introduced a C-terminal non-cleavable His6-tag and EcoRI (5′ end) and HindIII (3′ end) restriction sites by PCR amplification. The DNA generated was inserted into the pFastBac-1 plasmid, sequenced twice (both DNA strands) and introduced in E. coli XL1-blue for amplification. The hGBE1 p.Y329S mutant was generated from this recombinant plasmid by two sequential PCR reactions using Exact DNA polymerase (5 PRIME Co, Germany). The wild-type (WT) and p.Y329S hGBE1 cDNAs cloned in pFastBac-1 were introduced in E. coli DH10Bac competent cells, which contain the AcNPV (Autographa califormica nuclear polyhedrosis virus). The cDNAs were transferred from pFastBac-1 to the AcNPV bacmid by site-specific transposition. Finally, AcNPV bacmids containing full-length WT or p.Y329S GBE1 were purified and introduced into Sf9 insect cells Cellfectin (Invitrogen) as transfection agent. Full-length hGBE1 (WT and mutant) was purified similarly as with hGBE1trunc.

Using the assumption that the hGBE1-apo crystal structure represents an active enzyme conformation, the design of an hGBE1 p.Y329S stabilizing peptide was performed using a rigid backbone modelling of the mutation, in order to retain maximum similarity to the active enzyme.

In brief, a 17 Å grid was constructed at a 1 Å resolution in the solvent exposed region around position 329. Pepticom© ab initio peptide design algorithm was used to search for possible peptides within the grid which show favorable calculated binding affinities to the mutated GBE protein and reasonable solubility. The algorithm was supplemented by the Risk Adjusted Design algorithm (to be published separately), to generate a binding candidate ensemble. From the solution ensemble, a Leu-Thr-Lys-Glu (LTKE; SEQ ID NO: 1) peptide was selected for synthesis due to its calculated micromolar binding affinity, small size and the presence of a cationic lysine residue, which could increase the probability of cell membrane penetration via active transport. The peptide was synthesized using solid phase synthesis at a 98% level of purity.

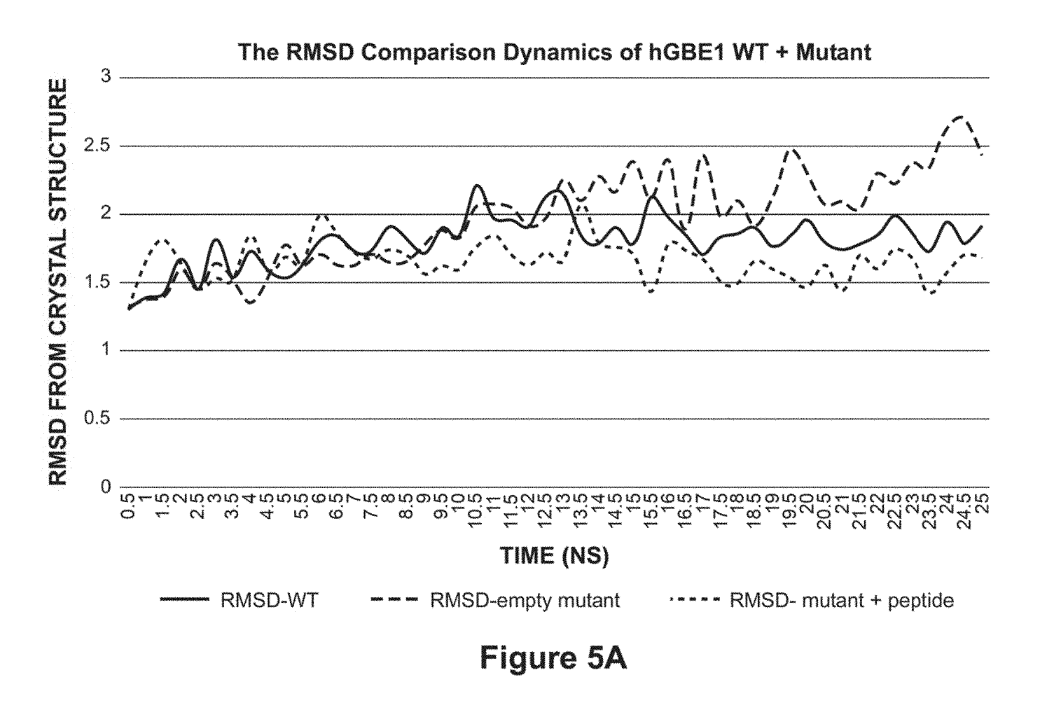

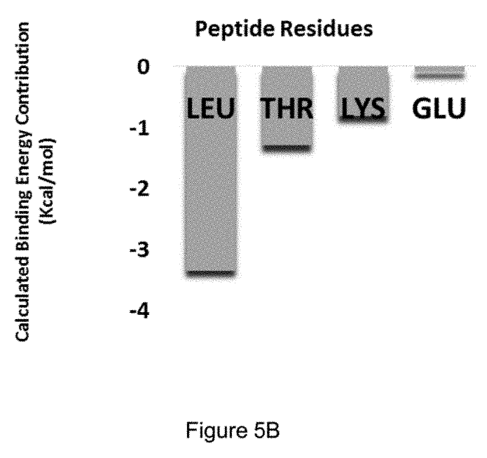

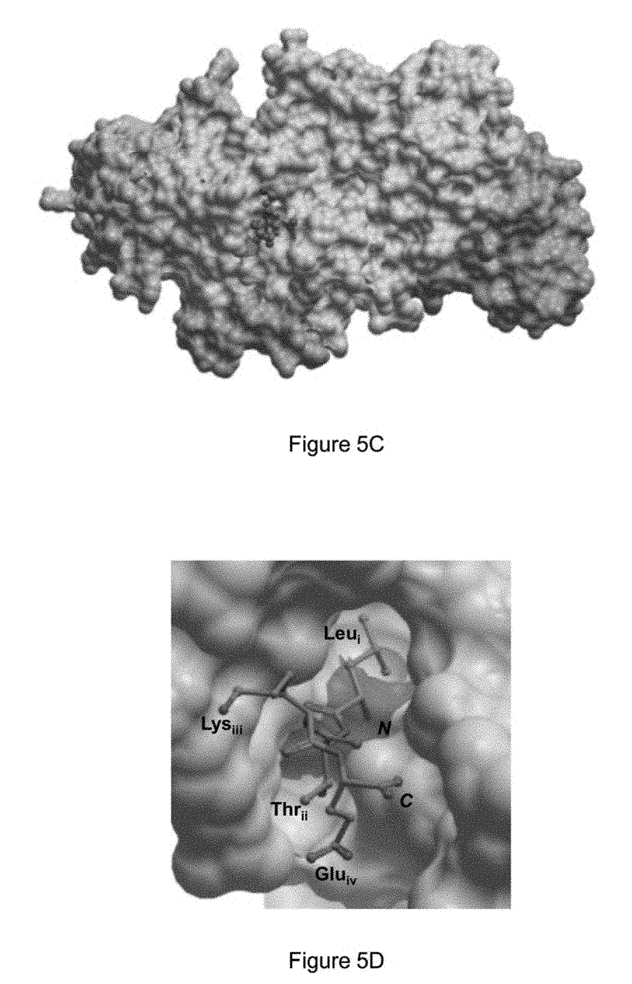

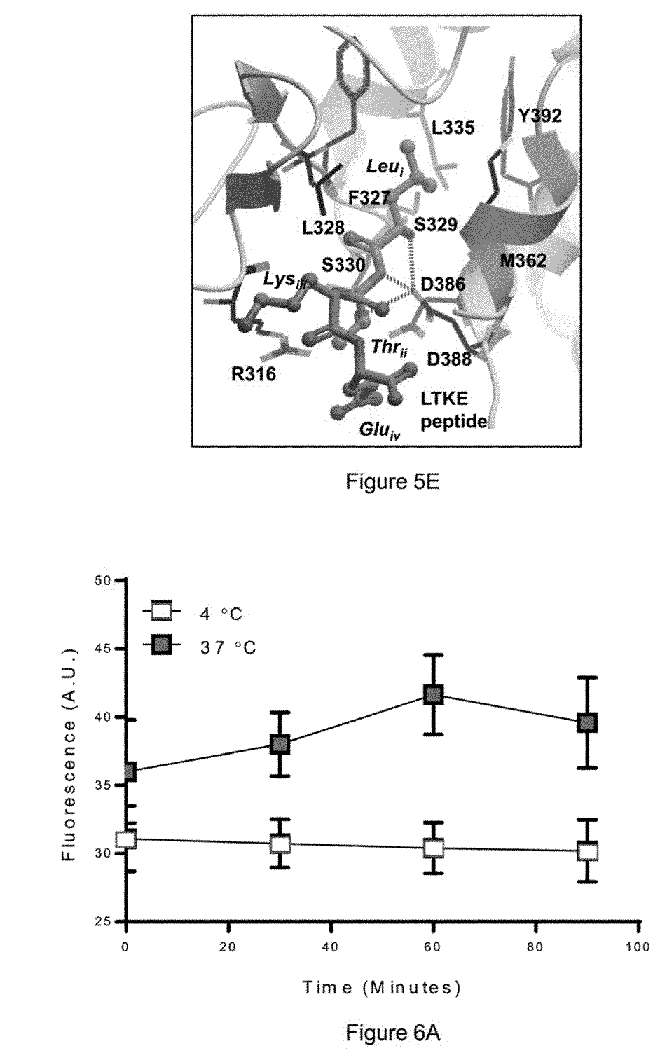

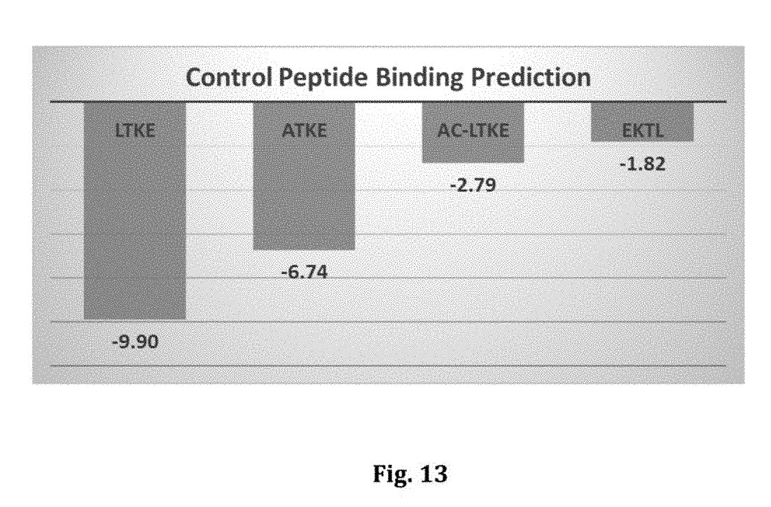

Screening around the solvent exposed Ser329ₘᵤₜₐₙₜ region in the aforementioned hGBE1-Y329S model, the ab initio peptide design algorithm gave as best hit a Leu-Thr-Lys-Glu (LTKE; SEQ ID NO: 1) peptide among the 6 top scores (Table 3) in terms of favourable binding affinities and solubility. Molecular dynamics simulation of wild-type hGBE1, hGBE1-Y329S, and LTKE-bound hGBE1-Y329S models indicated that LTKE stabilizes the mutated enzyme (FIG. 5A). Modelling of the LTKE peptide onto the model suggests that the N-terminal Leu (position i) is the primary contributor to peptide binding energy (FIG. 5B), with a calculated dissociation constant (K_{d}) of 1.6 μM (Table 3). Replacement of Leu at position i with Ala (ATKE peptide; SEQ ID NO: 8) or with acetyl-Leu (Ac-LTKE peptide) severely reduced peptide binding energy (FIG. 13), strongly suggesting a specific mode of action for the LTKE peptide. In the LTKE-bound hGBE1-Y329S model, the Leu side-chain can penetrate the cavity formed by the p.Y329S mutation (FIGS. 5C and 5D), recovering some of the hydrophobic interactions (e.g. with Phe327, Met362) offered by the wild-type tyrosyl aromatic ring, albeit with a different hydrogen bond pattern (FIG. 5E). The charged peptidyl N-terminus also forms hydrogen bonds with Ser329ₘᵤₜₐₙₜ, and forms a salt bridge with Asp386. The peptidyl Thr at position ii hydrogen bonds to Asp386, while the side-chains of Lys at position iii and Glu at position iv further provide long-range electrostatic interactions with hGBE1.

TABLE 3
Peptide ensemble analysis

| | Binding | Expected | | |
|---|---|---|---|---|
| | Free | Molar | | |
| | Energy* | Dissociation | Fills the | |
| Sequence (SEQ | (kcal/mol, | Constant | Y329S | |
| ID No.) | calculated) | (Kd)** | Space? | Model description |
| EKEPFEMFM (3) | −13.46 | 1.3 × 10⁻⁷ | NO | Primarily long range |
| | | | | electrostatics and hydrophobic |
| | | | | interactions. |
| LTKE (1) | −10.00 | 1.6 × 10⁻⁶ | YES | Hydrogen bond pattern combined |
| | | | | with hydrophobic interactions. |
| SSKI (4) | −9.46 | 2.4 × 10⁻⁶ | YES | Very similar model to 2, with |
| | | | | lower calculated affinity and less |
| | | | | optimal H-bond pattern. |
| MKWE (5) | −9.13 | 3.1 × 10⁻⁷ | Partially | Primarily long range |
| | | | | electrostatics and hydrophobic |
| | | | | interactions. |
| KSLRKW (6) | −8.57 | 4.6 × 10⁻⁶ | NO | Primarily long range |
| | | | | electrostatics and hydrophobic |
| | | | | interactions. |
| SDHRKMYEGR (7) | −8.26 | 5.8 × 10⁻⁶ | NO | A helical model primarily |
| | | | | composed of electrostatic |
| | | | | interactions. |

*Calculated using Pepticom's energy function, with the relationship to measured binding free energies: ΔGmeasured = (0.44)(ΔGcalculated) − 3.6 (based on the calculated to measured linear regression of 55 peptide-protein and protein-protein complexes with similar characteristics to the design parameters, R² = 0.47).
**Obtained using the equation: Kd = e^{ΔGi/RT} where ΔGi = (0.44)(ΔGcalculated) − 3.6 which serves as an estimate for the dissociation constant.

### Example 7—Peptide Rescue of hGBE1 p.Y329S

The potential of the LTKE peptide to rescue the destabilized mutant protein in vivo, was evaluated by testing it in APBD patient cells harboring the p.Y329S mutation.

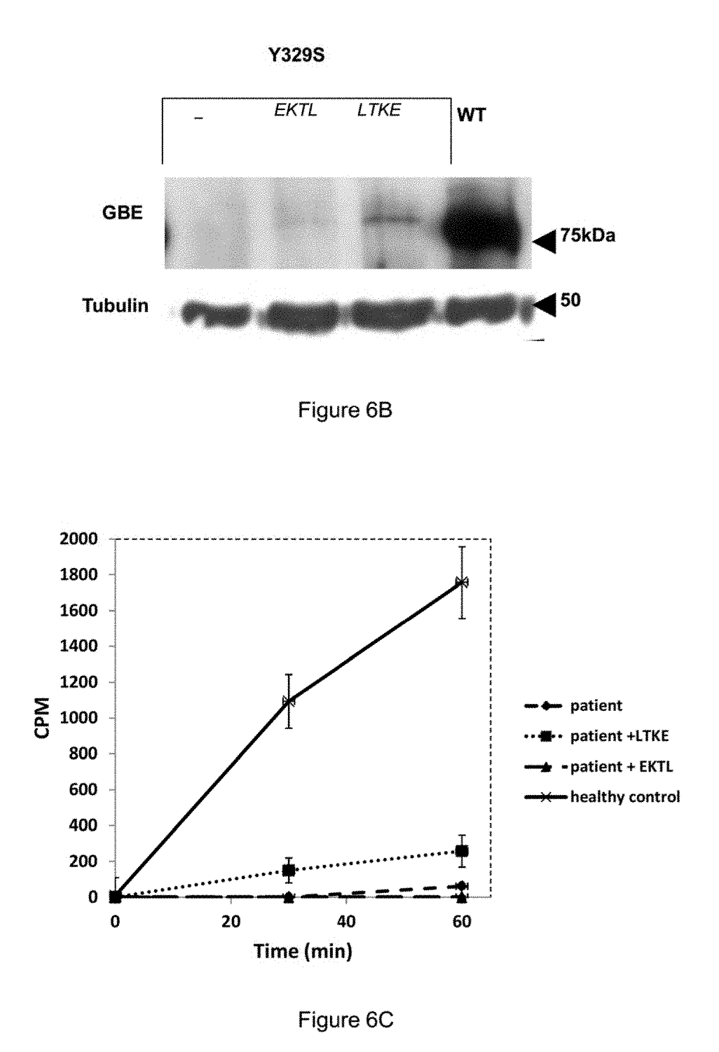

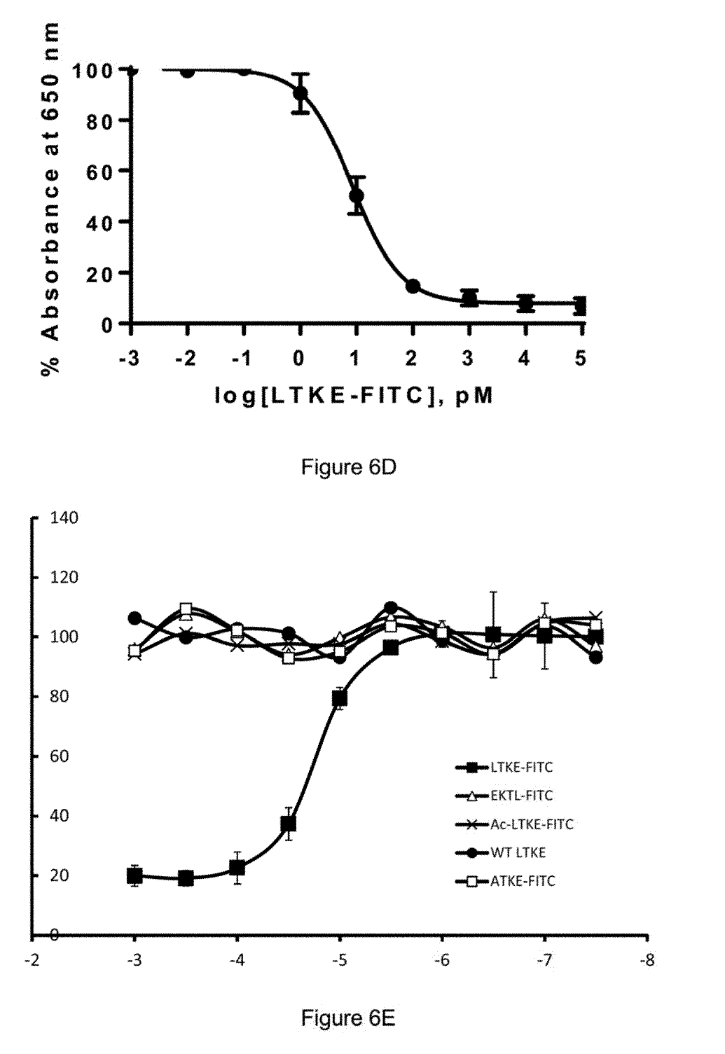

Binding of peptides to hGBE1 p.Y329S in intact fibroblasts was assessed by competitive hapten immuasssay. In brief, a standard curve was first generated to show that the immunoreactive LTKE-FITC peptide in solution can compete for HRP-conjugated FITC antibody binding with solid phase FITC. To generate the standard curve, plates coated overnight with 12.5 ng/ml BSA-FITC were incubated for 1 h at room temperature with an HRP conjugated anti-FITC antibody pretreated for 2 h with different concentrations of LTKE-FITC. The HRP substrate tetramethyl benzidine (TMB) was added for 0.5 h and absorbance at 650 nm was measured by the DTX 880 Multimode Detector. The resulting standard curve presented displacement of solid phase FITC by soluble LTKE-FITC (FIG. 6D). Curve was fit by non-linear regression using the 4 parameter logistic equation: % Absorbance (650 nm)=Bottom+(Top−Bottom)/(1+10̂((log(EC50−[LTKE−FITC])*Hillslope)), where Bottom=7.996, Top=100, EC50=8.460, Hillslope=−1.015. R²=0.9934.

Curve fitting, using the homologous one site competition model, was only found for APBD patient cells competed with LTKE-FITC (filled square, FIG. 6E). APBD patient cells competed with control peptides (i.e. ATKE-FITC, LTKE-FITC acetylated at the leucine (AcLTKE-FITC) or EKTL-FITC) did not demonstrate competitive binding (empty squares, crosses and triangles, respectively, FIG. 6E). In addition, wild type cells (i.e. cells that do not express the APBD mutation) did not demonstrate competitive binding of LTKE-FITC (circles, FIG. 6E). This competition model equation was: % Absorbance (650 nm)=(Bmax*[LTKE; SEQ ID NO: 1])/([LTKE; SEQ ID NO: 1]+peptide-FITC (M)+Kd)+Bottom where, Bmax=5229 nM, [LTKE; SEQ ID NO: 1]=316 nM, Kd=18 μM, Bottom=13.24 nM. R²=0.9458. In all experiments, cells from n=3 different APBD patients (or control unaffected subjects) were used. The results indicate that for obtaining competitive binding the cells must have the mutation and the peptide must include LTKE, and, optionally, a label, such as, FITC.

Upon establishment of competitive binding of the HRP-anti FITC antibody by the standard curve, the following cells were incubated with 316 nM LTKE peptide (SEQ ID NO: 1):
PBMCs isolated from APBD patients were incubated with FITC-labeled LTKE peptides at 37° C. (FIG. 6A, filled square) or 4° C. (FIG. 6A, empty squares). At the indicated times intracellular peptide uptake was determined by flow cytometry (FIG. 6A).
Isolated PBMCs from an APBD patient (Y329S), or a control subject (WT) were incubated overnight with or without the peptides indicated (20 μM). Lysed cells were subjected to SDS-PAGE and immunoblotting with anti-GBE1 and anti-α-tubulin (loading control) antibodies (FIG. 6B).
Isolated PBMCs were assayed for GBE activity (FIG. 6C).

To confirm that the peptide is internalized into cells, its sensitivity to uptake temperature in peripheral blood mononuclear cells (PBMCs) was determined. A time-dependent increase in the uptake of the C-terminal fluorescein isothiocyanate (FITC)-labelled peptide (LTKE-FITC) only at 37° C. and not at 4° C. was observed, suggesting it is actively transported into cells (FIG. 6A). Surprisingly, these peptide levels were sufficient to partially rescue mutant p.Y329S protein level in vivo as determined by Western blot analysis (FIG. 6B). Pre-incubation of PBMCs with the LTKE peptide (SEQ ID NO: 1) resulted in detectable mutant GBE1 protein, which was absent when the ‘reverse peptide’ (EKTL; SEQ ID NO: 2) was used, or in patient-derived cells with no peptide treatment. Unexpectedly, the LTKE (SEQ ID NO: 1) and LTKE-FITC peptides enhanced GBE1 activity by two fold, compared to untreated or EKTL-treated mutant cells, (>15% of unaffected control; FIG. 6C).

The hapten immunoassay (FIGS. 6D and 6E) showed that only the LTKE-FITC peptide, but not the FITC-labelled control peptides ATKE (SEQ ID NO: 8), Ac-LTKE (Ac-SEQ ID NO: 1) and EKTL with predicted inferior binding to hGBE1-Y329S model (FIG. 13), are able to out-compete LTKE (SEQ ID NO: 1) binding in patient skin fibroblasts. This competitive binding of LTKE (SEQ ID NO: 1), specific to mutant cells and to the peptide amino acid sequence, clearly indicates the binding specificity of the LTKE peptide (SEQ ID NO: 1) towards hGBE1 p.Y329S mutant. The apparent Kd of binding determined by the hapten immunoassay was 18 μM (FIG. 6E), within the range of error from the calculated Kd (1.6 μM; Table 3). Collectively, the data suggests that the LTKE peptide (SEQ ID NO: 1) may function as a stabilising chaperone for the mutant p.Y329S protein.

### Example 8—In Vivo Studies

APBD was first described as a clinicopathologic entity in 1971. It is characterized clinically by progressive upper and lower motor neuron dysfunction, marked distal sensory loss (mainly in the lower extremities), early neurogenic bladder, cerebellar dysfunction, and dementia. However, not all features are present in all affected individuals, especially early in the course. Neuropathologic findings reveal numerous large PG bodies in the peripheral nerves, cerebral hemispheres, basal ganglia, cerebellum, and spinal cord. Isolated cases of PG myopathy without peripheral nerve involvement have been described.

As disclosed herein, in APBD most common GBE1 mutation substitutes the 329th amino acid tyrosine with serine. Although tyrosine in this location is not required for enzymatic activity, it affects either proper folding of GBE or degradation of GBE in an unknown mechanism. Unexpectedly, as shown hereinabove and further disclosed in Froese et al. (ibid), a synthetic peptide LTKE (SEQ ID NO: 1) can restore the protein folding and increases GBE activity in the cells derived from APBD patients, by 2 folds.

Restoring enzyme activity with the synthetic peptide LTKE (SEQ ID NO: 1) is tested in APBD mouse model that carries the p.Y329S mutation, which LTKE (SEQ ID NO: 1) was designed to stabilize and increase the enzymatic activity. This mouse model has 16%, 21%, 21% and 37% GBE enzyme activity in muscle, heart, brain and liver, respectively, compared to wild type mice.

APBD mice are treated with a composition comprising the LTKE peptide (SEQ ID NO: 1). Compositions comprising 10, 20, 40 and 80 nmol doses of the peptide are administered intravenously. About 4 hours post administration, animals are sacrificed and GBE activity is determined in the following tissues: brain, heart, liver and muscle. The brain is of main interest since APBD mainly affects the neurons. In order to see 2 fold increase in the brain of the mouse model, which exhibits 21% enzyme activity, a change of about 50% changes in GBE activity has to be detected. Detection is carried by the method described in Froese et al. (ibid).

The dose that exhibits best GBE recovery is then administered to a new group of mice every 4, 8 and/or 16 hours for a period of 2, 4 or 8 days and for a long term period of six months. As a result, the optimum dose and half-life of the peptide or the stabilized protein is determined.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. An artificial peptide consisting of amino acid sequence Leu-Thr-Lys-Glu (SEQ ID NO:1).

2. (canceled)

3. A conjugate comprising the peptide of claim 1 and a moiety linked thereto, wherein the moiety is selected from the group consisting of a fluorescent probe, a photosensitizer, a chelating agent and a therapeutic agent.

4. The conjugate of claim 3, wherein the moiety is linked to the peptide via a spacer, and wherein the spacer is selected from the group consisting of a natural or non-natural amino acid, a short peptide having no more than 8 amino acids and a C₁-C₂₅ alkyl.

5. The conjugate of claim 4, wherein said moiety is a fluorescent probe.

6. The conjugate of claim 5, wherein said fluorescent probe is BPheide taurine amide (BTA), fluorenyl isothiocyanate (FITC), dansyl, rhodamine, eosin or erythrosine.

7-18. (canceled)

19. A method of treating a disease or disorder associated with glycogen storage in a subject in need thereof, the method comprising administering to said subject a pharmaceutical composition comprising an artificial peptide comprising the amino acid sequence set forth in SEQ ID NO: 1.

20. The method of claim 19, wherein the artificial peptide is consisting of the amino acid sequence set forth in SEQ ID NO: 1.

21. The method of claim 19, wherein the disease or disorder is glycogen storage disorder type IV (GSDIV) or the late-onset adult polyglucosan body disease (APBD).

22. The method of claim 19, wherein the pharmaceutical composition further comprises a moiety, the moiety being linked to the artificial peptide thereby forming a conjugate therewith, wherein the moiety is selected from the group consisting of a fluorescent probe, a photosensitizer, a chelating agent and a therapeutic agent.

23. The method of claim 22, wherein the moiety is linked to the peptide via a spacer, and wherein the spacer is selected from the group consisting of a natural or non-natural amino acid, a short peptide having no more than 8 amino acids and a C₁-C₂₅ alkyl.

24. The method of claim 22, wherein said moiety is a fluorescent probe.

25. The method of claim 24, wherein said fluorescent probe is BPheide taurine amide (BTA), fluorenyl isothiocyanate (FITC), dansyl, rhodamine, eosin or erythrosine.

26. (canceled)


SEQUENCE LISTING (68 sequences)

SEQ ID NO: 1 - protein, 4 aa, Artificial Sequence
  created in silico
LTKE

SEQ ID NO: 2 - protein, 4 aa, Artificial Sequence
  created in silico
EKTL

SEQ ID NO: 3 - protein, 9 aa, Artificial Sequence
  created in silico
EKEPFEMFM

SEQ ID NO: 4 - protein, 4 aa, Artificial Sequence
  created in silico
SSKI

SEQ ID NO: 5 - protein, 4 aa, Artificial Sequence
  created in silico
MKWE

SEQ ID NO: 6 - protein, 6 aa, Artificial Sequence
  in silico
KSLRKW

SEQ ID NO: 7 - protein, 10 aa, Artificial Sequence
  in silico
SDHRKMYEGR

SEQ ID NO: 8 - protein, 13 aa, Homo sapiens
NEGGEWAPGAEGV

SEQ ID NO: 9 - protein, 13 aa, Oryza sativa
HEGGEWAPAAQEA

SEQ ID NO: 10 - protein, 13 aa, Drosophila melanica
SEGGEWAPGARDV

SEQ ID NO: 11 - protein, 13 aa, Danio rerio
AEGTEWAPGAKAV

SEQ ID NO: 12 - protein, 13 aa, Mycobacterium tuberculosis
HLFAVWAPNAKGV

SEQ ID NO: 13 - protein, 13 aa, Escherichia coli
WLLSVWAPNARRV

SEQ ID NO: 14 - protein, 12 aa, Homo sapiens
DYGKWELWEILR

SEQ ID NO: 15 - protein, 12 aa, Oryza sativa
KFGIWSIWEVLR

SEQ ID NO: 16 - protein, 12 aa, Drosophila melanica
DFGKWELYEVLR

SEQ ID NO: 17 - protein, 12 aa, Danio rerio
EHGKWDLWEVLR

SEQ ID NO: 18 - protein, 12 aa, Mycobacterium tuberculosis
PSGVWELPEVRN

SEQ ID NO: 19 - protein, 12 aa, Escherichia coli
ESGIWELREVSN

SEQ ID NO: 20 - protein, 57 aa, Homo sapiens
TDSCYFHSGPRGTHDLWDSRLFAYSSWEILRFLLSNIRWWLEEYRFDGFRFDGVTSM

SEQ ID NO: 21 - protein, 55 aa, Escherichia coli
NLYEHSDPREGYHQDWNTLIYNYGRREVSNFLVGALYWIERFGIDALRVDAVASM

SEQ ID NO: 22 - protein, 54 aa, Mycobacterium tuberculosis
PLYEHSDPKRGEQLDWGTYVFDFGRPEVRNFLVANALYWQEFHIGLRVDAVASM

SEQ ID NO: 23 - protein, 59 aa, Saccharomyces cerevisiae
SDHQYFHSISSGRGEHPLWDSRLFNYGKFEVQRFLLANLAFYVDVYQFDGFRFDGVTSM

SEQ ID NO: 24 - protein, 56 aa, Salmonella typhi
HLYEHSDPREGYHQDWNTLIYNYGRREVSNYLVGNALYWMERFGIDALRVDAVASM

SEQ ID NO: 25 - protein, 56 aa, Shigella flexneri
NLYEHSDPREGYHQDWNTLIYNYGRREVSNFLVGNALYWIERFGIDALRVDAVASM

SEQ ID NO: 26 - protein, 58 aa, Bacillus anthracis
PTYEYKDKDVQENPVWGTTVNFDLGKEREVRNFLISNALFWMRYFHIDGFRVDAVANM

SEQ ID NO: 27 - protein, 48 aa, Arabidopsis thaliana
TDGQYFHSGSRGYHWMWDSRLFNYGSWEVLRYLLSNARWWLEEYKTSM

SEQ ID NO: 28 - protein, 56 aa, Mus musculus
TDSCYFHSGPRGTHDLWDSRLFIYSSWEVLRFLLSNIRWWLEEYCFDGFRDGVTSM

SEQ ID NO: 29 - protein, 58 aa, Oryza sativa
NTHESYFHTGDRGYHKLWDSRLFNYANWEVLRFLLSNLRYWMDEFMFDGFRFDGVTSM

SEQ ID NO: 30 - protein, 12 aa, Homo sapiens
GIIVLLDVVHSH

SEQ ID NO: 31 - protein, 12 aa, Oryza sativa
GLRVLMDVVHSH

SEQ ID NO: 32 - protein, 12 aa, Mycobacterium tuberculosis
GIGVIVDWVPAH

SEQ ID NO: 33 - protein, 12 aa, Escherichia coli
GLNVILDWVPGH

SEQ ID NO: 34 - protein, 12 aa, Homo sapiens
GVRIYVDAVINH

SEQ ID NO: 35 - protein, 12 aa, Bacillus amyloliquefaciens
NVQVYGDVVLNH

SEQ ID NO: 36 - protein, 10 aa, Homo sapiens
YRFDGFRFDG

SEQ ID NO: 37 - protein, 10 aa, Oryza sativa
FMFDGFRFDG

SEQ ID NO: 38 - protein, 10 aa, Mycobacterium tuberculosis
FHIDGLRVDA

SEQ ID NO: 39 - protein, 10 aa, Escherichia coli
FGIDALRVDA

SEQ ID NO: 40 - protein, 10 aa, Homo sapiens
IGVAGFRLDA

SEQ ID NO: 41 - protein, 10 aa, Bacillus amyloliquefaciens
LSLDGFRIDA

SEQ ID NO: 42 - protein, 8 aa, Homo sapiens
SITIAEDV

SEQ ID NO: 43 - protein, 8 aa, Oryza sativa
ATIVAEDV

SEQ ID NO: 44 - protein, 8 aa, Mycobacterium tuberculosis
IVTIAEES

SEQ ID NO: 45 - protein, 8 aa, Escherichia coli
AVTMAEES

SEQ ID NO: 46 - protein, 8 aa, Homo sapiens
PFIYQEVI

SEQ ID NO: 47 - protein, 8 aa, Bacillus amyloliquefaciens
MGTVAEYW

SEQ ID NO: 48 - protein, 9 aa, Homo sapiens
CIAYAESHD

SEQ ID NO: 49 - protein, 9 aa, Oryza sativa
CIAYAESHD

SEQ ID NO: 50 - protein, 8 aa, Mycobacterium tuberculosis
YVLPLSHD

SEQ ID NO: 51 - protein, 8 aa, Escherichia coli
FVLPLSHD

SEQ ID NO: 52 - protein, 9 aa, Homo sapiens
ALVFVDNHD

SEQ ID NO: 53 - protein, 9 aa, Bacillus amyloliquefaciens
SVTFVDNHD

SEQ ID NO: 54 - protein, 50 aa, Homo sapiens
PDSITIAEDVSGMPALCSPISQGGGGFDYRLAMAIPDKWIQLLKEFKDED

SEQ ID NO: 55 - protein, 50 aa, Oryza sativa
PEATIVAEDVSGMPVLCRPVDEGGVGFDFRLAMAIPDRWIDYLKNKEDRK

SEQ ID NO: 56 - protein, 49 aa, Mycobacterium tuberculosis
PGIVTIAEESTPWSGVTRPTNIGGLGFSMKWNMGWMHDTLDYVSRDPVY

SEQ ID NO: 57 - protein, 49 aa, Escherichia coli
SGAVTMAEESTDFPGVSRPQDMGGLGFWYKWNLGWMHDTLDYMKLDPVY

SEQ ID NO: 58 - protein, 42 aa, Homo sapiens
SKPFIYQEVIDLGGEPIKSSDYFGNGRTEFKYGAKLGTVIRK

SEQ ID NO: 59 - protein, 48 aa, Bacillus amyloliquefaciens
KEMFTVAEYWQNNAGKLENYLNKTSFNQSVFDVPLHFNLQAASSQGGG

SEQ ID NO: 60 - protein, 749 aa, Homo sapiens
MAAPMTPAARPEDYEAALNAALADVPELARLLEIDPYLKPYAVDFQRRYKQFSQILKNIGENEGGIDKFSRGYESFGVHRCADGGLYCKEWAPGAEGVFLTGDFNGWNPFSYPYKKLDYGKWELYIPPKQNKSVLVPHGSKLKVVITSKSGEILYRISPWAKYVVREGDNVNYDWIHWDPEHSYEFKHSRPKKPRSLRIYESHVGISSHEGKVASYKHFTCNVLPRIKGLGYNCIQLMAIMEHAYYASFGYQITSFFAASSRYGTPEELQELVDTAHSMGIIVLLDVVHSHASKNSADGLNMFDGTDSCYFHSGPRGTHDLWDSRLFAYSSWEILRFLLSNIRWWLEEYRFDGFRFDGVTSMLYHHHGVGQGFSGDYSEYFGLQVDEDALTYLMLANHLVHTLCPDSITIAEDVSGMPALCSPISQGGGGFDYRLAMAIPDKWIQLLKEFKDEDWNMGDIVYTLTNRRYLEKCIAYAESHDQALVGDKSLAFWLMDAEMYTNMSVLTPFTPVIDRGIQLHKMIRLITHGLGGEGYLNFMGNEFGHPEWLDFPRKGNNESYHYARRQFHLTDDDLLRYKFLNNFDRDMNRLEERYGWLAAPQAYVSEKHEGNKIIAFERAGLLFIFNFHPSKSYTDYRVGTALPGKFKIVLDSDAAEYGGHQRLDHSTDFFSEAFEHNGRPYSLLVYIPSRVALILQNVDLPNMAAPMTPAARPEDYEAALNAALADVPELARLLEIDPYLKPYAVDFQR

SEQ ID NO: 61 - protein, 702 aa, Mus musculus
MAAPAAPAAGETGPDARLEAALADVPELARLLEIDPYLKPFAADFQRRYKKFSQVLHDIGENEGGIDKFSRGYESFGIHRCSDGGIYCKEWAPGAEGVFLTGEFSGWNPFSHPYKKLEYGKWELYIPPKQNKSPLIPHGSKLKVVITSKSGEILYRISPWAKYVVRENNNVNYDWIHWAPEDPYKFKHSRPKKPRSLRIYESHVGISSHEGKIASYKHFTSNVLPRIKDLGYNCIQLMAIMEHAYYASFGYQITSFFAASSRYGTPEELKELVDTAHSMGIVVLLDVVHSHASKNSEDGLNMFDGTDSCYFHSGPRGTHDLWDSRLFIYSSWEVLRFLLSNIRWWLEEYCFDGFRFDGVTSMLYHHHGMGQGFSGDYNEYFGLQVDEDALIYLMLANHLAHTLYPDSITIAEDVSGMPALCSPTSQGGGGFDYRLAMAIPDKWIQLLKEFKDEDWNMGNIVYTLTNRRYLEKCVAYAESHDQALVGDKTLAFWLMDAEMYTNMSVLAPFTPVIDRGIQLHKMIRLITHGLGGEGYLNFMGNEFGHPEWLDFPRKGNNESYHYARRQFNLTDDDLLRYKFLNNFDRDMNRLEERCGWLSAPQAYVSEKHEANKTITFERAGLLFIFNFHPSKSYTDYRVGTATPGKFKIVLDSDAAEYGGHQRLDHNTNYFAEAFEHNGRPYSLLVYIPSRVALILQNVDLQN

SEQ ID NO: 62 - protein, 896 aa, Gallus gallus
MPSESRRCARLRAAQRPAERRRNLSQRSHPVPLPGSPSVAARTTRSPGLPPRPRPRGTRRAGSGGGGGRLSVPGLPPPEVRGKSEVLEDYLFIFKVIVKELCGDVTTSAINHFLAGAKIKVVRYALFYKRLKSIDDNEGGLNKFSKSYKSFGVNQFVDGGVYCKEWAPGAEAVFLTGDFNGWNPFSHPYKKMEYGKWELFIPPGQDGFSPVPHGSKLKVVIRAQNGELLYRISPWARYVVRYEGKVNYDWVHWDPPQSYIRKHRSPKKLKSLRIYESHVGIASPEGKIASYKNFTYNVLPRIRDLGYNCIQLMAVMEHAYYASFGYQVTSFFAASSRYGTPDDLKELIDVAHSMGITVLLDVVHSHASKNSEDGLNKFDGTDSCFFHSGPRGTHRIWDSRLFDYANWEVLRFLLSNLRMWIEDYGFDGFRFDGVTSMLYHDHGIGKEFSGDYNEYFGLDVDEDALCYLMLANHMINFLHPDCITIAEDVSGMPALCRPVAEGGGGFDYRLAMAIPDKWIKIIKELKDEDWNMGNIVYTLTNRRCDEKYIAYAESHDQALVGDKTLAFRLMDAEMYTNMSVFTPLTPVIDRGIQLHKMIRLITHTLGGDGYLNFMGNEFGHPEWLDFPRKGNNESFHYARRQFNLTDDHLLRYKFLNEFDRDMNKLEEKFGWLASPPAFVTEKHESNKVIAFERAGLLFIFNFHPYESYVDYRVGVEVPGKYKILMDSDASEYGGHQRLDHNTEYFSEEYPHNYRPNSVMQSNSQTSFSQHSRRCHKHLIKVYIPSRVAIVLQNTDVPQMPSESRRCARLRAAQRPAERRRNLSQRSHPVPLPGSPSVAARTTRSPGLPPRPRPRGTRRAGSGGGGGRLSVPGLPPPEVRGKSEVLEDYLFIFKVIV

SEQ ID NO: 63 - protein, 718 aa, Xenopus laevis
MATELGTVEVPELDVLLGQDPYLKPYEKDFHRRYRLFDRLLKSIEGNEGGLEKFSRSYQSFGVHVLENGGICCKEWAPGAEAMFLTGDFNGWNPFSHPYKKLDYGKWELHIPPREDKSVIIPHGSKLKVVMTSKSGETLYRISPWAKYVIREDNKAVYDWVHWEPPQPYKRKHASPKKLKSVRIYEAHVGIASSEGRIASYKNFTDNVLPKIKDLGYNCIQMMAIMEHAYYASFGYQITSFFAASSRYGTPDELKELIDVAHSMGIQVLLDVVHSHASNNTEDGLNKFDGTDSCFFHDGARGIHALWDSRLFDYSNWEVLRFLLSNLRWWIEEYGFDGFRFDGVTSMLYHHHGIGCGFSGGYNEYFGLHVDEDSLLYLLLANHMIHTLYPHCITVAEEVSGMPAICCPISQGGVGFDYRLAMAVPDKWIQILKELKDEDWDMGNIVHTLTNRRYNEKCIAYAESHDQALVGDKSLAFWLMDAEMYTNMSVFSPLTPVIDRGMQLHKMLRLITHALGGEGYLNFIGNEFGHPEWLDFPRKGNGESYHYARRQFHLIDDQQLRYRFLYAFDRDMNKLEEKFGWLAAPQAYISAKHESDKIIAFERANLIFIFNFHPYKSYTGYRVAVNKPGKYMIALDTDSSEYGGHQRINHKTEFFAEDAPYNSCSHSILVYIPCRVAIVLCNIDNSMATELGTVEVPELDVLLGQDPYLKPYEKDFHR

SEQ ID NO: 64 - protein, 721 aa, Danio rerio
MAESESNISVPHLKSLLQMDPYLKPFEKDFERRYGLFEKQLAFLEEAEGGFDHFTRSYESFGVQRLQDNSLVFKEWAPAAEALFLTGDFNGWDKFSHPYAKKEFGKWELHIPPKEDKTPAVTHNSKLKVVVHTSAGERLYRISPWAKYVTRHEKSVIYDWVHWDPPQPYIHKHPRPQKPRSLRIYESHVGIASPEGKIASYSNFTHNVLPRIKDLGYNSIQLMAIMEHAYYASFGYQVTSFFAASSRYGTPEELKELIDVAHSLGIIVLLDVVHSHASKNTEDGLNLFDGSDSCFFHSGPRGEHSLWDSRLFNYSSWEVLRFLLSNLRWWMEEYKFDGFRFDGVTSMLYHHHGIGSGFSGDYNEYFGLQVDEDSLVYLMLANHILHTLYEDCITIAEDVSGMPTLCCPVQQGGLGFDYRLAMAIPDKWIQILKEFKDEDWNMGNIVFTMTNRRYGEKCIAYAESHDQALVGDKTLAFWLMDKEMYTSMSALIPMNSIIDRGIQLHKMIRLLTHSLGGEGYLNFMGNEFGHPEWLDFPRIGNNESYHYARRQYNLVDMDHLRYRQLYAFDRDMNRTEDNYGWLAAPPAYVSVKHEGDKVIVFERANLIFIFNFHPFNSYSDYRVAVGPAGKYKIKLDSDEIQYGGHGRLDHNTEFFTEAMGLNNRPNSMMVYIPCRTAIVLANEEIDYCYMAESESNISVPHLKSLLQMDPYLKPFEKDFER

SEQ ID NO: 65 - protein, 711 aa, Drosophila melanogaster
MAEAKDIEKLFETDGYLRPFEHEIRRRHGVLEDWLNKINQSEGGLDGFSTAYKHYGLHFQPDNSVIAREWAPGARDVYLTGDFNNWHWESHPFKKLDFGKWELHLPPNEDGSPAIKHLSEIKIIIRNHSGQLLDRLSPWAKYVVQPPKSANQGVNYKQYVWEPPSYERYQRQHPGPPRPKSLRIYECHVGIASQEPRVGSYDEFADRIVPRIKRQGYNCIQVMAIMEHAYYASFGYQVTSFYAASSRYGNPEQLKRMIDVAHSHGLFVLLDVVHSHASKNVQDGLNQFDGTNSCFFHDGARGEHSLWDSRLFNYVEYEVLRFLLSNLRWWHDEYNFDGYRFDGVTSMLYHSRGIGEGFSGDYNEYFGLNVDTDALNYLGLANHLLHTIDSRIITIAEDVSGMPTLCRPVSEGGIGFDYRLGMAIPDKWIELLKEQSDDEWDMGNLVHTLTNRRWMENTVAYAESHDQALVGDKTIAFWLMDKEMYTHMSTLSDSSVIIDRGLALHKMIRLITHALGGEAYLNFMGNEFGHPEWLDFPRVGNNDSYHYARRQWNLVDDDLLKYKYLNEFDRAMNEAEERYGWLHSGPAWVSWKHEGDKIIAFERAGLVFVFNFHPQQSFTGYRVGTNWAGTYQAVLSSDDPLFGGHNRIDANCKHPSNPEGYAGRSNFIEVYTPSRTAVVYARVSDMAEAKDIEKLFETDGYLRPFEHEIRR

SEQ ID NO: 66 - protein, 796 aa, Oryza sativa
MLCLTSSSSSAPAPLLPSLADRPSPGIAGGGGNVRLSVVSSPRRSWPGKVKTNFSVPATARKNKTMVTVVEEVDHLPIYDLDPKLEEFKDHFNYRIKRYLDQKCLIEKHEGGLEEFSKGYLKFGINTVDGATIYREWAPAAQEAQLIGEFNNWNGAKHKMEKDKFGIWSIKISHVNGKPAIPHNSKVKFRFRHGGGAWVDRIPAWIRYATFDASKFGAPYDGVHWDPPACERYVFKHPRPPKPDAPRIYEAHVGMSGEEPEVSTYREFADNVLPRIRANNYNTVQLMAIMEHSYYASFGYHVTNFFAVSSRSGTPEDLKYLVDKAHSLGLRVLMDVVHSHASNNVTDGLNGYDVGQNTHESYFHTGDRGYHKLWDSRLFNYANWEVLRFLLSNLRYWMDEFMFDGFRFDGVTSMLYHHHGINKGFTGNYKEYFSLDTDVDAIVYMMLANHLMHKLLPEATIVAEDVSGMPVLCRPVDEGGVGFDFRLAMAIPDRWIDYLKNKEDRKWSMSEIVQTLTNRRYTEKCIAYAESHDQSIVGDKTIAFLLMDKEMYTGMSDLQPASPTINRGIALQKMIHFITMALGGDGYLNFMGNEFGHPEWIDFPREGNNWSYDKCRRQWSLVDTDHLRYKYMNAFDQAMNALEEEFSFLSSSKQIVSDMNEKDKVIVFERGDLVFVFNFHPNKTYKGYKVGCDLPGKYRVALDSDALVFGGHGRVGHDVDHFTSPEGMPGVPETNFNNRPNSFKVLSPPRTCVAYYRVDEDREELRRMVTVVEEVDHLPIYDLDPKLEEFKDHFNY

SEQ ID NO: 67 - protein, 709 aa, Caenorhabditis elegans
MVNTRPPKIDELLKIDPYLHDFQDEISRRYGVFLDYQRRIEECGGMEEFTSSYKQFGLNVQPDNSVKGLEWAPAAEKLALIGDFNNWDQNANVYKKEEHGKWSITVPAKEDGSCPIPHNSVIKIAVSRHGATHFKLSPWATFVTCPNPKETVIYHQNFWNPPEKYQLKEARPARPASLRIYEAHVGISSSEGKINTYREFADDVLPRIQKQGYNAIQLMAVMEHVYYASFGYQVSNFFAVSSRCGTPEDLKYLVDKAHSLGIFMLLDVVHSHASKNVEDGLNQWDGSNGGYFHDNARGYHNLWDSRLFDYTQTETLRFLLSNVRWWVEEYGFDGFRFDGVSSMIYHSHGMNDDFCGGYPMYFGLNADTDSLVYLMLANDFLHKKYPFMITIAEEVSGMPGICRPVEEGGQGFDYRLAMALPDMWIKILKHTSDEDWKIDDIVFNLENRRYAEKHVAYAESHDQALVGDKTIAFWLMDKEMYDFMSTDSPLTPIIDRGLSLHKLIRLITIGLGGEAWLNFIGNEFGHPEWLDFPRVGNGESFHYARRQFNLVDAEYLRYKFLNNWDREMMLLEERTGFLHKGYAYTSWKHDGDKTIVFERGGLVFVINLHPTKSFADYSIGVNTPGRYRIALNSDESKFGGHNRIDNSIKFHTTDDGYAGRRHRLQVYITCRTAIVLEKEDDMVNTRPPKIDELLKIDPYLHDFQDEISR

SEQ ID NO: 68 - protein, 617 aa, Escherichia coli
MLSEGTHLRPYETLGAHADTMDGVTGTRFSVWAPNARRVSVVGQFNYWDGRRHPMRLRKESGIWELFIPGAHNGQLYKYEMIDANGNLRLKSDPYAFEAQMRPETASLICGLPEKVVQTEERKKANQFDAPISIYEVHLGSWRRHTDNNFWLSYRELADQLVPYAKWMGFTHLELLPINEHPFDGSWGYQPTGLYAPTRRFGTRDDFRYFIDAAHAAGLNVILDWVPGHFPTDDFALAEFDGTNLYEHSDPREGYHQDWNTLIYNYGRREVSNFLVGNALYWIERFGIDALRVDAVASMIYRDYSRKEGEWIPNEFGGRENLEAIEFLRNTNRILGEQVSGAVTMAEESTDFPGVSRPQDMGGLGFWYKWNLGWMHDTLDYMKLDPVYRQYHHDKLTFGILYNYTENFVLPLSHDEVVHGKKSILDRMPGDAWQKFANLRAYYGWMWAFPGKKLLFMGNEFAQGREWNHDASLDWHLLEGGDNWHHGVQRLVRDLNLTYRHHKAMHELDFDPYGFEWLVVDDKERSVLIFVRRDKEGNEIIVASNFTPVPRHDYRFGINQPGKWREILNTDSMHYHGSNAGNGGTVHSDEIASHGRQHSLSLTLPPLATIWLVREAE